(19) United States
(12) Patent Application Publication
(10) Pub. No.: US 2007/0014841 A1
(43) Pub. Date: Jan. 18, 2007

(54) Pharmaceutical compositions

(76) Inventors: Denis Martin, St-Augustin-de-Desmaures (CA); Stephane Rioux, Ohain (BE)

Correspondence Address: MILLEN, WHITE, ZELANO & BRANIGAN, PC, 2200 CLARENDON BLVD, SUITE 1400, ARLINGTON, VA, 22201, US

(21) Appl. No.: 11/349,553

(22) Filed: Feb. 8, 2006

Related U.S. Application Data
(60) Provisional application No. 60/650,543, filed on Feb. 8, 2005.

(51) Int. Cl. A61K 39/09; A61K 9/127

(52) U.S. Cl. 424/450; 424/244.1; 424/190.1

(57) ABSTRACT

The present invention relates to polypeptides, epitopes and antibodies directed to these epitopes, more particularly to the Sip polypeptide of Group B Streptococcus (GBS), also called Streptococcus agalactiae which may be used to prevent, diagnose and/or treat streptococcal infection.

This application claims the benefit of the filing date of U.S. Provisional Application Ser. No. 60/650,543 filed Feb. 8, 2005.

### BACKGROUND OF THE INVENTION

Streptococcus are gram (+) bacteria that are differentiated by group specific carbohydrate antigens A through 0 found on their cell surface. Streptococcus groups are further distinguished by type-specific capsular polysaccharide antigens. Several serotypes have been identified for the Group B Streptococcus (GBS): Ia, Ib, II, III, IV, V, VI, VII and VIII. GBS also contains several antigenic proteins, such as “c-proteins” (α, β and γ), Rib, R and C5a peptidase, some of which have been characterized.

Although GBS is a common component of the normal human vaginal and colonic flora this pathogen has long been recognized as a major cause of neonatal sepsis and meningitis, late-onset meningitis in infants, postpartum endometritis as well as mastitis in dairy herds. Expectant mothers exposed to GBS are at risk of postpartum infection and may transfer the infection to their baby as the child passes through the birth canal. Although the organism is sensitive to antibiotics, the high attack rate and rapid onset of sepsis in neonates and meningitis in infants results in high morbidity and mortality.

GBS infections in infants are restricted to very early infancy. Approximately 80% of infant infections occur in the first days of life, so-called early-onset disease. Late-onset infections occur in infants between 1 week and 3 months of age. Clinical syndromes of GBS disease in newborns include sepsis, meningitis, pneumonia, cellulitis, osteomyelitis, septic arthritis, endocarditis, epiglottis. In addition to acute illness due to GBS, which is itself costly, GBS infections in newborns can result in death, disability, and, in rare instances, recurrence of infection. Although the organism is sensitive to antibiotics, the high attack rate and rapid onset of sepsis in neonates and meningitis in infants results in high morbidity and mortality.

Among pregnant women, GBS causes clinical illness ranging from mild urinary tract infection to life-threatening sepsis and meningitis, including also osteomyelitis, endocarditis, amniotis, endometritis, wound infections (postcesarean and postepisiotomy), cellulitis, fasciitis.

Among non-pregnant adults, the clinical presentations of invasive GBS disease most often take the form of primary bacteremia but also skin of soft tissue infection, pneumonia, urosepsis, endocarditis, peritonitis, meningitis, empyema. Skin of soft tissue infections include cellulitis, infected peripheral ulcers, osteomyelitis, septic arthritis and decubiti or wound infections. Among people at risk, there are debilitated hosts such as people with a chronic disease such as diabetes mellitus, cancer or elderly people.

GBS infections can also occur in animals and cause mastitis in dairy herds.

To find a vaccine that will protect individuals from GBS infection, researchers have turned to the type-specific antigens. These polysaccharides have proven to be poorly immunogenic in humans and are restricted to the particular serotype from which the polysaccharide originates. These capsular polysaccharides, when coupled to carrier protein such as tetanus toxoid, were shown to be more immunogenic in preclinical assays and human clinical trials, and as expected the protection was shown to be type specific. Based on the current information on serotype distribution, a conjugate vaccine would have to include types Ia, Ib, II, III and V to prevent the majority of disease in North America. However, the formulation would have to be modified to be effective in other parts of the world, such as Japan, where other serotypes, such as VI and VIII, are more prevalent.

An alternative strategy for protecting neonates and infants would be to develop a GBS vaccine based on an ubiquitous protein. Bacterial surface proteins have numerous advantages for vaccine development. Indeed, such bacterial proteins were shown for other bacterial pathogens to be present in most pathogenic strains and to induce cross-protective immunity. Furthermore, these proteins do not need to be conjugated to other molecules, since they elicit an effective T-cell dependent antibody response resulting in long-term immunity. GBS surface proteins already being investigated as potential vaccine candidates are the R protein, α and β sub-units of the c protein, and the Rib protein. All these proteins are capable of eliciting antibodies in mice and to some extent prolong life and protect against lethal bacterial challenges.

PCT WO 99/42588 has been published Feb. 17, 1999 entitled ‘Group B Streptococcus antigens’ describing the polypeptide which is claimed to be antigenic. This polypeptide is now known under the name Sip, for Surface immunogenic protein (Brodeur et al., 2000, Infect. Immun. 68:5610).

This polypeptide was found to be highly conserved and produced by every GBS examined to date, which included representative isolates of all serotypes (Brodeur et al.). This 53-kDa polypeptide is recognized by the human immune system. More importantly, immunization of adult mice with the Sip-polypeptide was shown to induce a strong specific antibody response and to confer protection against experimental infection with GBS strains representing serotypes Ia/c, Ib, II/R, III, V and VI (Brodeur et al.). It was also demonstrated that Sip-specific antibodies recognized their epitopes at the cell surfaces of different GBS strains, which included representatives of all nine serotypes (Rioux et al., 2001, Infect. Immun. 69:5162). In addition, it was recently reported that passive administration of rabbit anti-Sip serum to pregnant mice or immunization of female mice before pregnancy with purified recombinant Sip conferred protective immunity to their offsprings against GBS infection (Martin et al., 2002, Infect. Immun. 70:4897).

Therefore there remains a need for Group B Streptococcus polypeptides that may be used to prevent, diagnose and/or treat Group B Streptococcus infection.

### BRIEF DESCRIPTION OF THE DRAWINGS

FIG. 1 represents the DNA sequence of sip gene from serotype Ia/c Group B Streptococcus strain C388/90; (SEQ ID NO: 1).

FIG. 2 represents the amino acid sequence of Sip polypeptide from serotype Ia/c Group B Streptococcus strain C388/90; (SEQ ID NO: 2).

FIG. 3 represents the DNA sequence of c1sip gene; (SEQ ID NO: 3).

FIG. 4 represents the amino acid sequence of C1rSip polypeptide; (SEQ ID NO: 4).

FIG. 5 represents the DNA sequence of c2sip gene; (SEQ ID NO: 5).

FIG. 6 represents the amino acid sequence of C2rSip polypeptide; (SEQ ID NO: 6).

FIG. 7 represents the DNA sequence of sip gene from serotype Ia/c GBS strain C388/90 without the region coding for its leader peptide; (SEQ ID NO: 7).

FIG. 8 represents the amino acid sequence Sip polypeptide from serotype Ia/c GBS strain C388/90 without the 25 amino acid residues leader peptide; (SEQ ID NO: 8).

FIG. 9 represents the DNA sequence of c1sip gene without the region coding for its leader peptide; (SEQ ID NO: 9).

FIG. 10 represents the amino acid sequence C1rSip polypeptide without the 25 amino acid residues leader peptide; (SEQ ID NO: 10).

FIG. 11 represents the DNA sequence of c2sip gene without the region coding for its leader peptide; (SEQ ID NO: 11).

FIG. 12 represents the amino acid sequence C2rSip polypeptide without the 25 amino acid residues leader peptide; (SEQ ID NO: 12).

FIG. 13 depicts the comparison of the nucleotide sequences of the Sip gene (SEQ ID NO: 1) from the Group B Streptococcus serotype Ia/c C388/90 strain and the genes encoding C1Sip (SEQ ID NO: 3) and C2Sip (SEQ ID NO: 5) polypeptides. Identical nucleotides are presented as *.

FIG. 14 depicts the comparison of the predicted amino acid sequences of the Sip polypeptide (SEQ ID NO: 2) from the Group B Streptococcus serotype Ia/c C388/90 strain and the C1rSip (SEQ ID NO: 4) and C2rSip (SEQ ID NO: 6) polypeptides. Identical amino acid residues are presented as *.

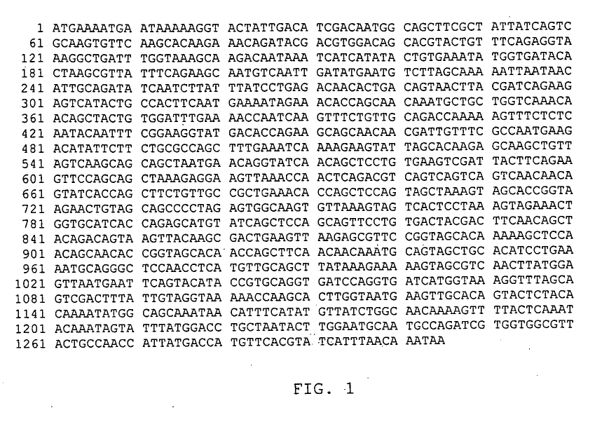

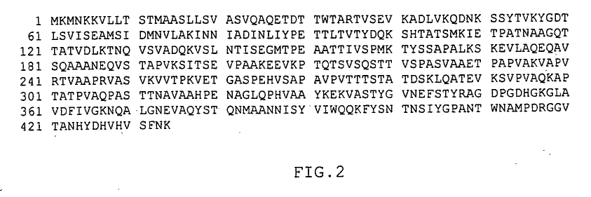

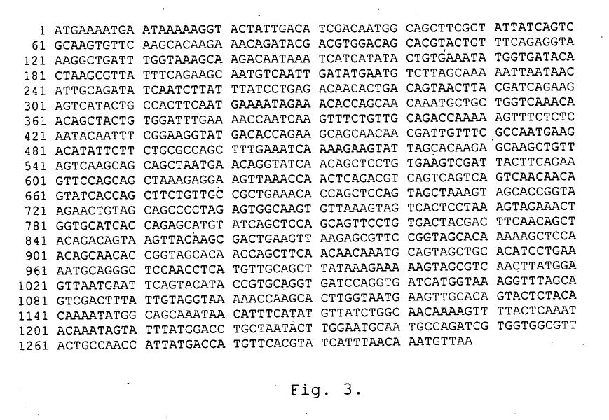

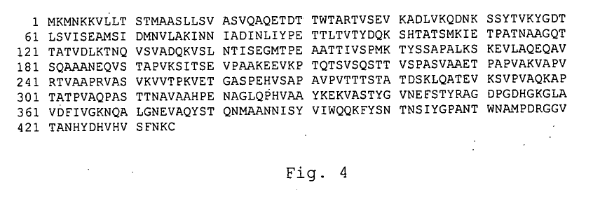

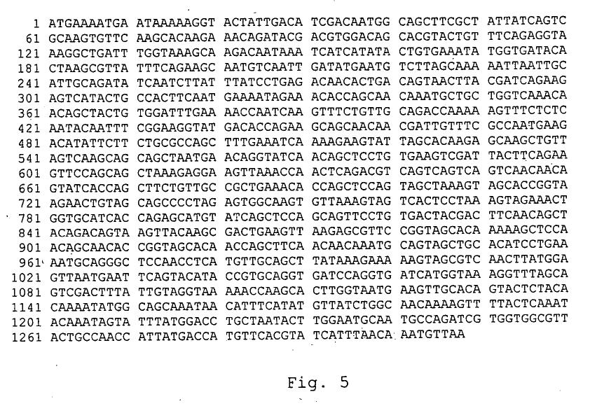

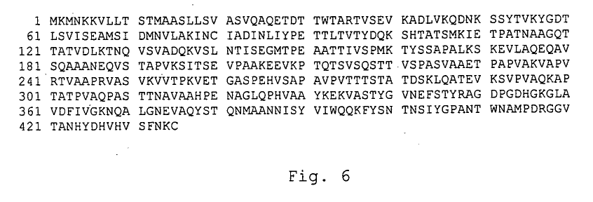

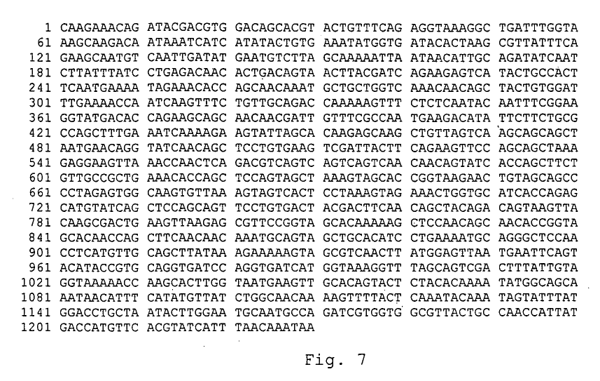

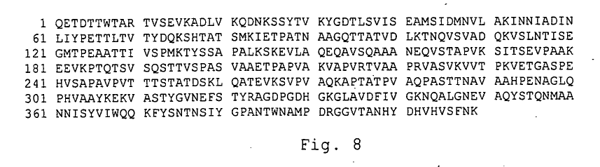

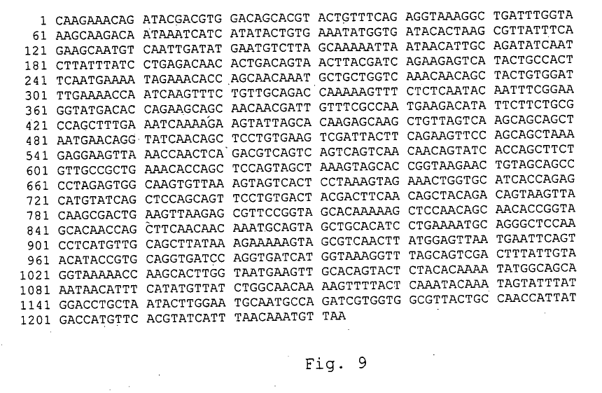

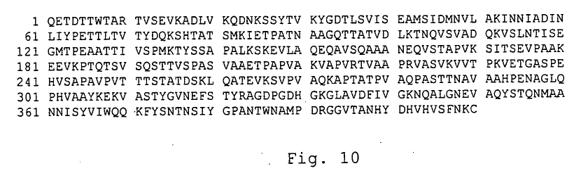

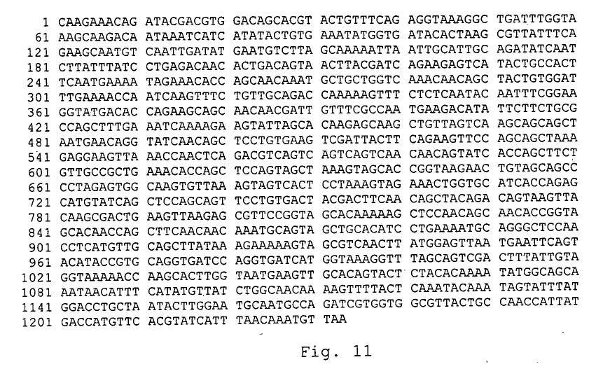

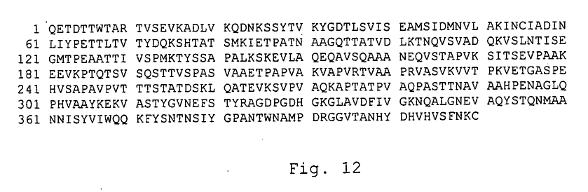

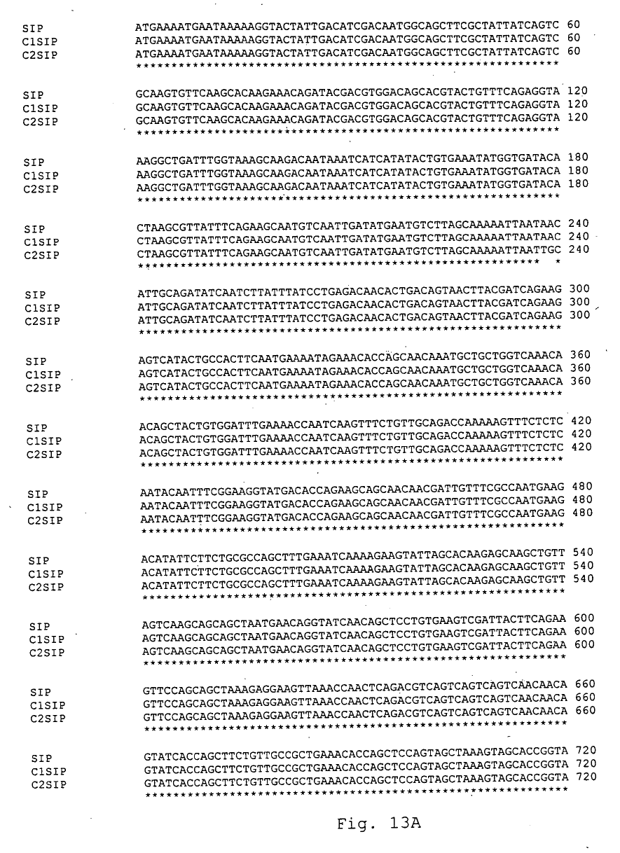

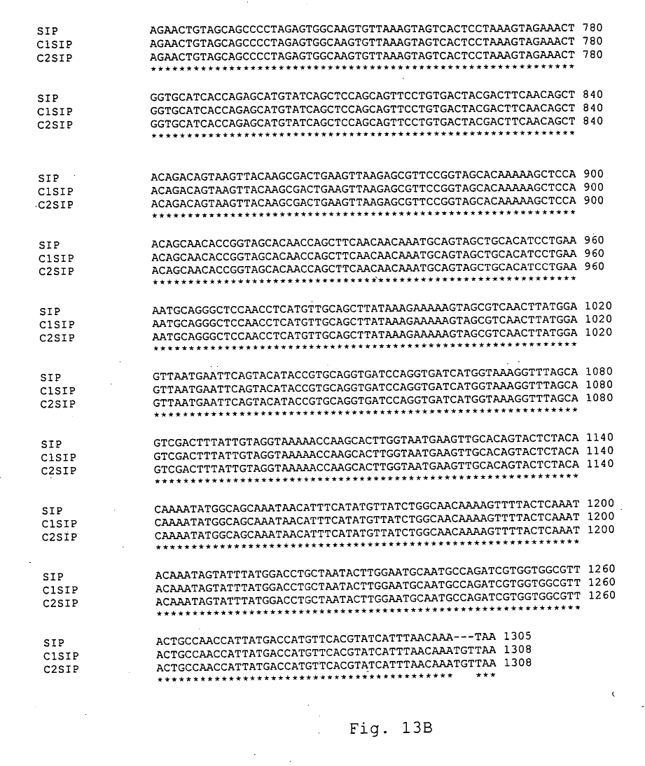

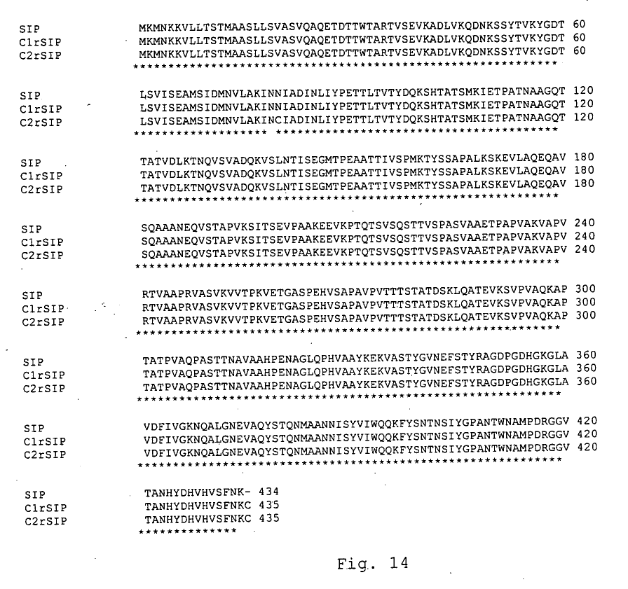

### DETAILED DESCRIPTION OF THE INVENTION

The present invention provides polypeptides, more particularly the Sip polypeptide of Group B Streptococcus (GBS), also called Streptococcus agalactiae and pharmaceutical compositions comprising a liposome associated to these antigens, polypeptides, epitopes or antibodies directed to these epitopes, or corresponding DNA fragments which may be used to prevent, diagnose and/or treat streptococcal infection.

According to one aspect, the present invention relates to pharmaceutical compositions comprising a liposome associated with polypeptides comprising SEQ ID No: 2, 4, 6, 8, 10, 12 or fragments or analogs thereof.

According to one aspect, the present invention relates to pharmaceutical compositions comprising a liposome associated with polypeptides comprising SEQ ID No: 2, 4, 6, 8, 10 or 12.

According to one aspect, the present invention relates to pharmaceutical compositions comprising a liposome associated with epitope bearing portions of a polypeptide comprising SEQ ID No: 2, 4, 6, 8, 10, 12 or fragments or analogs thereof.

According to one aspect, the present invention relates to pharmaceutical compositions comprising a liposome associated with epitope bearing portions of a polypeptide comprising SEQ ID No: 2, 4, 6, 8, 10 or 12.

According to one aspect, the present invention provides a pharmaceutical compositions comprising a liposome associated with an isolated polypeptide chosen from:
(a) a polypeptide having at least 70% identity to a second polypeptide comprising SEQ ID No: 2, 4, 6, 8, 10, 12 or fragments or analogs thereof;
(b) a polypeptide having at least 80% identity to a second polypeptide comprising SEQ ID No: 2, 4, 6, 8, 10, 12 or fragments or analogs thereof;
(c) a polypeptide having at least 95% identity to a second polypeptide comprising SEQ ID No: 2, 4, 6, 8, 10, 12 or fragments or analogs thereof;
(d) a polypeptide having at least 98% identity to a second polypeptide comprising SEQ ID No: 2, 4, 6, 8, 10, 12 or fragments or analogs thereof;
(e) a polypeptide having at least 99% identity to a second polypeptide comprising SEQ ID No: 2, 4, 6, 8, 10, 12 or fragments or analogs thereof;
(f) a polypeptide comprising SEQ ID No: 2, 4, 6, 8, 10, 12 or fragments or analogs thereof;
(g) a polypeptide capable of raising antibodies having binding specificity for a polypeptide comprising SEQ ID No: 2, 4, 6, 8, 10, 12 or fragments or analogs thereof;
(h) an epitope bearing portion of a polypeptide comprising SEQ ID No: 2, 4, 6, 8, 10, 12 or fragments or analogs thereof;
(i) the polypeptide of (a), (b), (c), (d), (e), (f), (g) or (h) wherein the N-terminal Met residue is deleted;
(j) the polypeptide of (a), (b), (c), (d), (e), (f), (g), (h) or (i) wherein the secretory amino acid sequence is deleted.

According to one aspect, the present invention provides a pharmaceutical compositions comprising a liposome associated with an isolated polypeptide chosen from:
(a) a polypeptide having at least 70% identity to a second polypeptide comprising SEQ ID No: 2, 4, 6, 8, 10 or 12;
(b) a polypeptide having at least 80% identity to a second polypeptide comprising SEQ ID No: 2, 4, 6, 8, 10 or 12;
(c) a polypeptide having at least 95% identity to a second polypeptide comprising SEQ ID No: 2, 4, 6, 8, 10 or 12;
(d) a polypeptide having at least 98% identity to a second polypeptide comprising SEQ ID No: 2, 4, 6, 8, 10 or 12;
(e) a polypeptide having at least 99% identity to a second polypeptide comprising SEQ ID No: 2, 4, 6, 8, 10 or 12;
(f) a polypeptide comprising SEQ ID No: 2, 4, 6, 8, 10 or 12;
(g) a polypeptide capable of raising antibodies having binding specificity for a polypeptide comprising SEQ ID No: 2, 4, 6, 8, 10 or 12;
(h) an epitope bearing portion of a polypeptide comprising SEQ ID No: 2, 4, 6, 8, 10 or 12;
(i) the polypeptide of (a), (b), (c), (d), (e), (f), (g) or (h) wherein the N-terminal Met residue is deleted;
(j) the polypeptide of (a), (b), (c), (d), (e), (f), (g), (h) or (i) wherein the secretory amino acid sequence is deleted.

As used herein, “associated with” means that the polypeptides of the invention are preferably covalently linked to the lipids to confer attachment of the polypeptides to the liposomes.

In a further embodiment, the polypeptides present in the pharmaceutical compositions comprising a liposome associated with polypeptides in accordance with the present invention are antigenic and therefore the pharmaceutical compositions are antigenic.

In a further embodiment, the polypeptides present in the pharmaceutical compositions comprising a liposome associated with polypeptides in accordance with the present invention are immunogenic and therefore the pharmaceutical compositions are immunogenic.

In a further embodiment, the polypeptides present in the pharmaceutical compositions comprising a liposome associated with polypeptides in accordance with the present invention can elicit an immune response in a host and therefore the pharmaceutical compositions can elicit an immune response in a host. In a further embodiment, the immune response is systemic. In a further embodiment, the immune response is mucosal.

In a further embodiment, the present invention also relates to pharmaceutical compositions comprising a liposome associated with polypeptides which are able to raise antibodies having binding specificity to the polypeptides of the present invention as defined above.

An antibody that “has binding specificity” is an antibody that recognizes and binds the selected polypeptide but which does not substantially recognize and bind other molecules in a sample, e.g., a biological sample, which naturally includes the selected peptide. Specific binding can be measured using an ELISA assay in which the selected polypeptide is used as an antigen.

In accordance with the present invention, “protection” in the biological studies is defined as an increase in the survival rate which is itself defined as an increase in the number of mice surviving a GBS deadly challenge comparatively to control mice.

In an additional aspect of the invention there are provided pharmaceutical compositions comprising a liposome associated with antigenic and/or immunogenic fragments of the polypeptides of the invention, or of analogs thereof.

The fragments of the present invention should include one or more such epitopic regions or be sufficiently similar to such regions to retain their antigenic and/or immunogenic properties. Thus, for fragments according to the present invention the degree of identity is perhaps irrelevant, since they may be 100% identical to a particular portion of a polypeptide or analog thereof as described herein. The present invention further provides an immunogenic fragment of a polypeptide of the invention, said fragment being a contiguous portion of the polypeptide of the invention. The present invention further provides fragments having at least 10 contiguous amino acid residues from the polypeptide sequences of the present invention. In one embodiment, at least 15 contiguous amino acid residues. In one embodiment, at least 20 contiguous amino acid residues. In one embodiment, at least 30 contiguous amino acid residues. In one embodiment, at least 40 contiguous amino acid residues. In one embodiment, at least 50 contiguous amino acid residues. In one embodiment, at least 100 contiguous amino acid residues. In one embodiment, at least 150 contiguous amino acid residues. In one embodiment, at least 200 contiguous amino acid residues. In one embodiment, at least 250 contiguous amino acid residues. In one embodiment, at least 300 contiguous amino acid residues. In one embodiment, at least 350 contiguous amino acid residues. In one embodiment, at least 400 contiguous amino acid residues.

The present invention further provides a fragment which has the same or substantially the same immunogenic activity as the polypeptide comprising Seq. ID no. 2, 4, 6, 8, 10 or 12. The fragment (when coupled to a carrier, if necessary) is capable of raising an immune response which recognizes the Sip polypeptide.

Such an immunogenic fragment may include, for example, the Sip polypeptide lacking an N-terminal leader peptide, and/or any putative structural domains.

The present invention further provides a fragment of Sip comprising substantially all of the extra cellular domain of a polypeptide which has at least 70% identify, preferably 80% identity, more preferably 95% identity, to a second polypeptide comprising Seq. ID No. 2, 4, 6, 8, 10 or 12, over the entire length of said sequence.

The present invention further provides pharmaceutical compositions comprising a liposome associated with fragments which comprise a B-cell or T-helper epitope.

The present invention further provides pharmaceutical compositions comprising a liposome associated with a fragment that may be part of a larger polypeptide. It can be advantageous to include an additional amino acid sequence which contains secretory or leader sequences, or sequences which aid in purification such as multiple histidine residues, or an additional sequence which increases stability during recombinant production, or an additional polypeptide which increases the immunogenic potential of the final polypeptide.

The skilled person will appreciate that pharmaceutical compositions Comprising a liposome associated with analogs of the polypeptides of the invention will also find use in the context of the present invention, i.e. as antigenic/immunogenic material. Thus, for instance proteins or polypeptides which include one or more additions, deletions, substitutions or the like are encompassed by the present invention.

As used herein, “fragments”, “analogs” or “derivatives” of the polypeptides of the invention include those polypeptides in which one or more of the amino acid residues are substituted with a conserved or non-conserved amino acid residue (preferably conserved) and which may be natural or unnatural. In one embodiment, derivatives and analogs of polypeptides of the invention will have about 70% identity with those sequences illustrated in the figures or fragments thereof. That is, 70% of the residues are the same. The % identity can either be over the whole sequence or the region to which it corresponds or matches. In a further embodiment, polypeptides will have greater than 70% identity. In a further embodiment, polypeptides will have greater than 80% identity. In a further embodiment, polypeptides will have greater than 85% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

These substitutions are those having a minimal influence on the secondary structure and hydropathic nature of the polypeptide. Preferred substitutions are those known in the art as conserved, i.e. the substituted residues share physical or chemical properties such as hydrophobicity, size, charge or functional groups. These include substitutions such as those described by Dayhoff, M. in Atlas of Protein Sequence and Structure 5, 1978 and by Argos, P. in EMBO J. 8, 779-785, 1989. For example, amino acids, either natural or unnatural, belonging to one of the following groups represent conservative changes:

ala, pro, gly, gln, asn, ser, thr, val;
cys, ser, tyr, thr;
val, ile, leu, met, ala, phe;
lys, arg, orn, his;
and
phe, tyr, trp, his.

The preferred substitutions also include substitutions of D-enantiomers for the corresponding L-amino acids.

The percentage of homology is defined as the sum of the percentage of identity plus the percentage of similarity or conservation of amino acid type.

In one embodiment, analogs of polypeptides of the invention will have about 70% identity with those sequences illustrated in the figures or fragments thereof. That is, 70% of the residues are the same. In a further embodiment, polypeptides will have greater than 80% identity. In a further embodiment, polypeptides will have greater than 85% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

In one embodiment, analogs of polypeptides of the invention will have about 70% homology with those sequences illustrated in the figures or fragments thereof. In a further embodiment, polypeptides will have greater than 80% homology. In a further embodiment, polypeptides will have greater than 85% homology. In a further embodiment, polypeptides will have greater than 90% homology. In a further embodiment, polypeptides will have greater than 95% homology. In a further embodiment, polypeptides will have greater than 99% homology. In a further embodiment, analogs of polypeptides of the invention will have fewer than about 20 amino acid residue substitutions, modifications or deletions and more preferably less than 10.

One can use a program such as the CLUSTAL program to compare amino acid sequences. This program compares amino acid sequences and finds the optimal alignment by inserting spaces in either sequence as appropriate. It is possible to calculate amino acid identity or homology for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

It is well known that it is possible to screen an antigenic polypeptide to identify epitopic regions, i.e. those regions which are responsible for the polypeptide's antigenicity or immunogenicity. Methods for carrying out such screening are well known in the art. Thus, the fragments of the present invention should include one or more such epitopic regions or be sufficiently similar to such regions to retain their antigenic/immunogenic properties.

Thus, what is important for analogs, derivatives or fragments is that they possess at least a degree of the antigenicity/immunogenicity of the protein or polypeptide from which they are derived.

Furthermore, in those situations where amino acid regions are found to be polymorphic, it may be desirable to vary one or more particular amino acids to more effectively mimic the different epitopes of the different GBS strains.

In a further embodiment, the present invention also relates to pharmaceutical compositions comprising a liposome associated with chimeric polypeptides which comprise one or more polypeptides or fragments or analogs thereof of the invention.

In a further embodiment, the present invention also relates to pharmaceutical compositions comprising a liposome associated with chimeric polypeptides comprising two or more polypeptides comprising SEQ ID No: 2, 4, 6, 8, 10, 12 or fragments or analogs thereof; provided that the polypeptides are linked as to formed a chimeric polypeptide.

In a further embodiment, the present invention also relates to pharmaceutical compositions comprising a liposome associated with chimeric polypeptides comprising two or more polypeptides comprising SEQ ID No: 2, 4, 6, 8, 10 or 12, provided that the polypeptides are linked as to form a chimeric polypeptide.

Preferably, a fragment, analog or derivative of a polypeptide of the pharmaceutical compositions of the invention will comprise at least one antigenic region i.e. at least one epitope.

In a particular embodiment, polypeptide fragments and analogs comprised in the pharmaceutical compositions of the invention do not contain a starting residue, such as methionine (Met) or valine (Val). Preferably, polypeptides will not incorporate a leader or secretory sequence (signal sequence). The signal portion of a polypeptide of the invention may be determined according to established molecular biological techniques. In general, the polypeptide of interest may be isolated from a GBS culture and subsequently sequenced to determine the initial residue of the mature protein and therefore the sequence of the mature polypeptide.

It is understood that polypeptides for the pharmaceutical compositions of the invention can be produced and/or used without their start codon (methionine or valine) and/or without their leader peptide to favor production and purification of recombinant polypeptides. It is known that cloning genes without sequences encoding leader peptides will restrict the polypeptides to the cytoplasm of E. coli and will facilitate their recovery (Glick, B. R. and Pasternak, J. J. (1998) Manipulation of gene expression in prokaryotes. In “Molecular biotechnology: Principles and applications of recombinant DNA”, 2nd edition, ASM Press, Washington D.C., p. 109-143).

The Sip polypeptide was shown to be antigenically highly conserved and present at the surface of intact GBS cells where it is easily accessible to specific antibodies. In addition, the mature Sip protein can also be found in the culture supernantant after growth of the GBS cells. Coupling of the Sip polypeptide to liposome vesicles was found to improve the specific immune response.

Liposomes are made of phospholipids and other polar amphiles, which form closed concentric bilayer membranes (summarized in Gregoriades, G., Immunology Today, 11, 3, 89 (1990); Lasic, D., American Scientist, 80, p. 20 (1992); Remington's on Pharmaceutical Sciences, 18th ed., 1990, Mack Publishing Co., Pennsylvania., p. 1691]. The primary constituent of liposomes are lipids, which have a polar hydrophilic “head” attached to a long, nonpolar, hydrophobic “tail”. The hydrophilic head typically consists of a phosphate group, while the hydrophobic tail is made of two long hydrocarbon chains. Since the lipid molecules have one part that is water-soluble and another part that is not, they tend to aggregate in ordered structures that sequester the hydrophobic tails from water molecules. In the process, liposomes can entrap water and solutes in their interior, or molecules with hydrophobic regions can also be incorporated directly into the liposomal membranes. Many phospholipids, alone or in combination, with other lipids will form liposomes. By convention, liposomes are categorized by size, and a 3-letter acronym is used to designate the type of liposome being discussed. Multilamellar vesicles are designated “MLV”, large unilamellar vesicles “LUV”, small unilamellar vesicles “SUV”. These designations are sometimes followed by the chemical composition of the liposome. Nomenclature and a summary of known liposomes is described in Storm et al, 1998, PSIT, 1:19-31. Liposomes are efficient adjuvant boosting the humoral as well as the cellular immune response against an antigen.

The invention provides pharmaceutical compositions comprising liposomes constituted from phospholipids. These phospholipids can be synthetized or extracted from bacterial cells, soybean, eggs.

The invention provides a process for covalently linking the recombinant Sip polypeptides onto different liposome formulations.

Liposomes can be prepared with various synthetic phospholipids (List 1) or bacterial phospholipids and/or cholesterol and/or DC-cholesterol, which can be combined at different ratios.

The liposomes of the invention can be prepared from a variety of vesicle-forming lipids including phosphatidyl ethers and esters, such as phosphatidylethanloamine (PE), phosphatidylserine (PS), phosphatidylglycerol (PG) and phosphatidylcholine (PC) but also from glycerides, such as dioleoylglycerosuccinate; cerebrosides; gangliosides, sphyngomyelin; steroids, such as cholesterol or DC-cholesterol; and other lipids, as well as excipients such as Vitamin E or Vitamin C palmitate.

List 1 provides a partial list of synthetic lipids that can be used to prepare Sip-liposome preparations. Other lipids can be used and are described in Remington's on Pharmaceutical Sciences, 18th ed., 1990, Mack Publishing Co., Pennsylvania, p. 390.

The Sip-liposome preparations of the invention are to be incorporated in pharmaceutical compositions.

List 1. List of synthetic lipids used to prepare Sip-liposome preparations.
1,2-Dilauroyl-sn-Glycero-3-Phosphate (DLPA),
Dimyristoyl-sn-Glycero-3-Phosphate (DMPA),
1,2-Dipalmitoyl-sn-Glycero-3-Phosphate (DPPA),
1,2-Distearoyl-sn-Glycero-3-Phosphate (DSPA),
1,2-Dioleoyl-sn-Glycero-3-Phosphate (DOPA),
1-Palmitoyl-2-Oleoyl-sn-Glycero-3-Phosphate (POPA),
1,2-Dilauroyl-sn-Glycero-3-Phosphocholine (DLPC),
1,2-Ditridecanoyl-sn-Glycero-3-Phosphocholine,
1,2-Dimyristoyl-sn-Glycero-3-Phosphocholine (DMPC),
1,2-Dipentadecanoyl-sn-Glycero-3-Phosphocholine,
1,2-Dipalmitoyl-sn-Glycero-3-Phosphocholine (DPPC),
1,2-Diheptadecanoyl-sn-Glycero-3-Phosphocholine,
1,2-Distearoyl-sn-Glycero-3-Phosphocholine (DSPC),
1,2-Dimyristoleoyl-sn-Glycero-3-Phosphocholine,
1,2-Dipalmitoleoyl-sn-Glycero-3-Phosphocholine,
1,2-Dioleoyl-sn-Glycero-3-Phosphocholine (DOPC),
1-Myristoyl-2-Palmitoyl-sn-Glycero-3-Phosphocholine,
1-Myristoyl-2-Stearoyl-sn-Glycero-3-Phosphocholine,
1-Palmitoyl-2-Myristoyl-sn-Glycero-3-Phosphocholine,
1-Palmitoyl-2-Stearoyl-sn-Glycero-3-Phosphocholine,
1-Palmitoyl-2-Oleoyl-sn-Glycero-3-Phosphocholine (POPC),
1-Palmitoyl-2-Linoleoyl-sn-Glycero-3-Phosphocholine,
1,2-Dilauroyl-sn-Glycero-3-Phosphoethanolamine (DLPE),
1,2-Dimyristoyl-sn-Glycero-3-Phosphoethanolamine (DMPE),
1,2-Dipalmitoyl-sn-Glycero-3-Phosphoethanolamine (DPPE),
1,2-Dipalmitoleoyl-sn-Glycero-3-Phosphoethanolamine,
1,2-Distearoyl-sn-Glycero-3-Phosphoethanolamine (DSPE),
1,2-Dioleoyl-sn-Glycero-3-Phosphoethanolamine (DOPE),
1-Palmitoyl-2-Oleoyl-sn-Glycero-3-Phosphoethanolamine (POPE),
1,2-Dilauroyl-sn-Glycero-3-[Phospho-RAC-(1-glycerol)] (DLPG),
1,2-Dimyristoyl-sn-Glycero-3-[Phospho-RAC-(1-glycerol)] (DMPG), 1,2-Dipalmitoyl-sn-Glycero-3-[Phospho-RAC-(1-glycerol)] (DPPG), 1,2-Distearoyl-sn-Glycero-3-[Phospho-RAC-(1-glycerol)] (DSPG),
1,2-Dioleoyl-sn-Glycero-3-[Phospho-RAC-(1-glycerol)] (DOPG), 1-Palmitoyl-2-Oleoyl-sn-Glycero-3-[Phospho-RAC-(1-glycerol)] (POPG),
1,2-Dilauroyl-sn-Glycero-3-[Phospho-L-Serine] (DLPS),
1,2-Dimyristoyl-sn-Glycero-3-[(Phospho-L-Serine] (DMPS),
1,2-Dipalmitoyl-sn-Glycero-3-[Phospho-L-Serine] (DPPS),
1,2-Distearoyl-sn-Glycero-3-[Phospho-L-Serine] (DSPS),
1,2-Dioleoyl-sn-Glycero-3-[Phospho-L-Serine] (DOPS),
1-Palmitoyl-2-Oleoyl-sn-Glycero-3-[Phospho-L-Serine] (POPS),
1,2-Dioleoyl-3-Trimethylammonium-Propane (DOTAP),
1,2-Dipalmitoyl-3-Trimethylammonium-Propane (DPTAP),
1,2-Distearoyl-3-Trimethylammonium-Propane (DSTAP),
1,2-Dimyristoyl-3-Trimethylammonium-Propane (DMTAP).

The fluidity and stability of the liposomal membrane will depend on the transition temperature (temperature at which hydrocarbon regions change from a quasicrystalline to a more fluid state) of the phospholipids.

Modifications of membrane fluidity, number of lamellae, vesicle size, surface charge, lipid to antigen ratio and localization of the antigen within the liposome can modulate the ajduvanticity of liposomal preparations.

The preparation of liposomes can be made by a number of different techniques including the incorporation of a lipid-modified-protein into pre-made liposomes permeabilized by the addition of detergent at sub-CMC concentrations, detergent dilution, ethanol injection; ether infusion; detergent removal; solvent evaporation; evaporation of organic solvents from chloroform in water emulsions; extrusion of multilamellar vesicles through a nucleopore polycarbonate membrane; freezing and thawing of phospholipid mixtures, as well as sonication and homogenization.

Lipids can be dissolved in a suitable organic solvent or mixture of organic solvents, such as a chloroform:methanol solution in a round bottom glass flask and dried using a rotatory evaporator to achieve an even film on the vessel.

Some liposome formulations can also be prepared with an adjuvant such as lipophilic molecules such as Lipid A, monophosphoryl lipid A (MPLA), lipopolysaccharide's such as QuilA, QS21, alum, MF59, p3CSS, MTP-PE, as well as water-soluble molecules, including cytokines such as interferons. In a preferred embodiment, the liposome composition comprises about 0-10% adjuvant(s). In a more preferred embodiment, the adjuvant is present in less than about 5%. The values may be vol/vol or wt/wt depending upon the adjuvant.

According to the present invention, the liposome plays a critical role in antigen delivery as the polypeptide-liposome composition is directly presented to the immune system following removal from the circulation by cells of the immune system. In addition, the choice of the immunostimulatory pathways can be altered by making changes to the lipid composition of the liposome. For example, different immunostimulatory molecules, such as Lipid A, muramyl di- and tripeptide-PE and cationic lipids can be formulated into the liposome.

The liposomes are efficient adjuvant boosting the humoral as well as the cellular immune response against an antigen. Modifications of membrane fluidity, number of lamellae, vesicle size, surface charge, lipid to antigen ratio and localization of the antigen within the liposome can modulate the adjuvanticity of liposomal preparations.

In a prefered embodiment, the lipid formulation contain between 0 and 50 mol % cholesterol and/or DC-cholesterol.

According to another aspect of the invention, there are also provided (i) a composition of matter containing a polypeptide of the invention, together with a liposome, carrier, diluent or adjuvant; (ii) a pharmaceutical composition comprising a polypeptide of the invention and a liposome, carrier, diluent or adjuvant; (iii) a vaccine comprising a polypeptide of the invention and a liposome, carrier, diluent or adjuvant; (iv) a method for inducing an immune response against GBS, in a host, by administering to the host, an immunogenically effective amount of a pharmaceutical composition of the invention to elicit an immune response, e.g., a protective immune response to GBS; and particularly, (v) a method for preventing and/or treating a GBS infection, by administering a prophylactic or therapeutic amount of a pharmaceutical composition of the invention or specific protective antibodies to a host in need.

According to another aspect, there are provided pharmaceutical compositions comprising a liposome, one or more GBS polypeptides of the invention in a mixture with a pharmaceutically acceptable adjuvant. Suitable adjuvants include (1) oil-in-water emulsion formulations such as MF59™, SAF™, Ribi™; (2) Freund's complete or incomplete adjuvant; (3) salts i.e. Alum, AlK(SO₄)₂, AlNa(SO₄)₂, AlNH₄(SO₄)₂, Al(OH)₃, AlPO₄, silica, kaolin, Alhydrogel™, Adjuphos™; (4) saponin derivatives such as Stimulon™ or particles generated therefrom such as ISCOMs (immunostimulating complexes); (5) cytokines such as interleukins, interferons, macrophage colony stimulating factor (M-CSF), tumor necrosis factor (TNF); (6) other substances such as carbon polynucleotides i.e. poly IC and poly AU, detoxified cholera toxin (CTB) and E. coli heat labile toxin for induction of mucosal immunity. A more detailed description of adjuvants is available in a review by M. Z. I Khan et al. in Pharmaceutical Research, vol. 11, No. 1 (1994) pp 2-11, and also in another review by Gupta et al., in Vaccine, Vol. 13, No. 14, pp 1263-1276 (1995) and in WO 99/24578. Preferred adjuvants include QuilA™, QS21™, Alhydrogel™ and Adjuphos™.

Pharmaceutical compositions of the invention may be administered parenterally by injection, rapid infusion, nasopharyngeal absorption, dermoabsorption, or buccal or oral.

The term pharmaceutical composition is also meant to include antibodies. In accordance with the present invention, there is also provided the use of one or more antibodies having binding specificity for the polypeptides of the present invention for the treatment or prophylaxis of GBS infection and/or diseases and symptoms mediated by GBS infection.

Pharmaceutical compositions of the invention are used for the prophylaxis of streptococcal infections and/or diseases and symptoms mediated by streptococcal infections as described in Manual of Clinical Microbiology, P. R. Murray (Ed, in chief), E. J. Baron, M. A. Pfaller, F. C. Tenover and R. H. Yolken. ASM Press, Washington, D.C. seventh edition, 1999, 1773p.

In one embodiment, pharmaceutical compositions of the present invention are used for the treatment or prophylaxis of endemic and epidemic diseases, such as neonatal sepsis and meningitis, late-onset meningitis in infants, postpartum endometritis as well as mastitis in dairy herds. In one embodiment, vaccine compositions of the invention are used for the treatment or prophylaxis of streptococcal infections and/or diseases and symptoms mediated by streptococcal infections. In a further embodiment, the streptococcal infection is GBS.

In a further embodiment, the invention provides a method for prophylaxis or treatment of GBS infection in a host susceptible to GBS infection comprising administering to said host a prophylactic or therapeutic amount of a composition of the invention.

As used in the present application, the term “host” includes mammals. In a further embodiment, the host is a dairy herd. In a further embodiment, the mammal is human. In a further embodiment, the host is an adult. In a further embodiment, the host is a pregnant woman. In a further embodiment, the host is a non-pregnant woman. In a further embodiment, the host is a neonate, infant or child.

In a particular embodiment, pharmaceutical compositions are administered to those hosts at risk of GBS infection such as neonates, infants, children, elderly and immunocompromised hosts.

In a particular embodiment, pharmaceutical compositions are administered to those hosts at risk of GBS infection such as adults.

Pharmaceutical compositions are preferably in unit dosage form of about 0.001 to 100 μg/kg (antigen/body weight) and more preferably 0.01 to 10 μg/kg and most preferably 0.1 to 1 μg/kg 1 to 3 times with an interval of about 1 to 6 week intervals between immunizations.

Pharmaceutical compositions are preferably in unit dosage form of about 0.1 μg to 10 mg and more preferably 1 μg to 1 mg and most preferably 10 to 100 μg 1 to 3 times with an interval of about 1 to 6 week intervals between immunizations.

According to another aspect, there is provided a process for producing polypeptides of the invention by recombinant techniques by expressing a polynucleotide encoding said polypeptide in a host cell and recovering the expressed polypeptide product.

Alternatively, the polypeptides can be produced according to established synthetic chemical techniques i.e. solution phase or solid phase synthesis of oligopeptides which are ligated to produce the full polypeptide (block ligation).

General methods for obtention and evaluation of polynucleotides and polypeptides are described in the following references: Sambrook et al, Molecular Cloning: A Laboratory Manual, 2nd ed, Cold Spring Harbor, N.Y., 1989; Current Protocols in Molecular Biology, Edited by Ausubel F. M. et al., John Wiley and Sons, Inc. New York; PCR Cloning Protocols, from Molecular Cloning to Genetic Engineering, Edited by White B. A., Humana Press, Totowa, N.J., 1997, 490 pages; Protein Purification, Principles and Practices, Scopes R. K., Springer-Verlag, New York, 3rd Edition, 1993, 380 pages; Current Protocols in Immunology, Edited by Coligan J. E. et al., John Wiley & Sons Inc., New York.

The present invention provides a process for producing a polypeptide comprising culturing a host cell of the invention under conditions suitable for expression of said polypeptide.

For recombinant production, host cells are transfected with vectors which encode the polypeptides of the invention, and then cultured in a nutrient media modified as appropriate for activating promoters, selecting transformants or amplifying the genes. Suitable vectors are those that are viable and replicable in the chosen host and include chromosomal, non-chromosomal and synthetic DNA sequences e.g. bacterial plasmids, phage DNA, baculovirus, yeast plasmids, vectors derived from combinations of plasmids and phage DNA. The polypeptide sequence may be incorporated in the vector at the appropriate site using restriction enzymes such that it is operably linked to an expression control region comprising a promoter, ribosome binding site (consensus region or Shine-Dalgarno sequence), and optionally an operator (control element). One can select individual components of the expression control region that are appropriate for a given host and vector according to established molecular biology principles (Sambrook et al, Molecular Cloning: A Laboratory Manual, 2nd ed, Cold Spring Harbor, N.Y., 1989; Current Protocols in Molecular Biology, Edited by Ausubel F. M. et al., John Wiley and Sons, Inc. New York). Suitable promoters include but are not limited to LTR or SV40 promoter, E. coli lac, tac or trp promoters and the phage lambda P_{L} promoter. Vectors will preferably incorporate an origin of replication as well as selection markers i.e. ampicilin resistance gene. Suitable bacterial vectors include pET, pQE70, pQE60, pQE-9, pD10 phagescript, psiX174, pbluescript SK, pbsks, pNH8A, pNH16a, pNH18A, pNH46A, ptrc99a, pKK223-3, pKK233-3, pDR540, pRIT5 and eukaryotic vectors pBlueBacIII, pWLNEO, pSV2CAT, pOG44, pXT1, pSG, pSVK3, pBPV, pMSG and pSVL. Host cells may be bacterial i.e. E. coli, Bacillus subtilis, Streptomyces; fungal i.e. Aspergillus niger, Aspergillus nidulins; yeast i.e. Saccharomyces or eukaryotic i.e. CHO, COS.

Upon expression of the polypeptide in culture, cells are typically harvested by centrifugation then disrupted by physical, or chemical means (if the expressed polypeptide is not secreted into the media) and the resulting crude extract retained to isolate the polypeptide of interest. Purification of the polypeptide from culture media or lysate may be achieved by established techniques depending on the properties of the polypeptide i.e. using ammonium sulfate or ethanol precipitation, acid extraction, anion or cation exchange chromatography, phosphocellulose chromatography, hydrophobic interaction chromatography, hydroxylapatite chromatography and lectin chromatography. Final purification may be achieved using HPLC.

The polypeptides may be expressed with or without a leader or secretion sequence. In the former case the leader may be removed using post-translational processing (see U.S. Pat. No. 4,431,739; U.S. Pat. No. 4,425,437; and U.S. Pat. No. 4,338,397) or be chemically removed subsequent to purifying the expressed polypeptide.

According to a further aspect, the pharmaceutical compositions of the invention may be used in a diagnostic test for streptococcal infection, in particular GBS infection.

Several diagnostic methods are possible, for example detecting GBS organism in a biological sample, the following procedure may be followed:
a) obtaining a biological sample from a host;
b) incubating an antibody or fragment thereof reactive with a pharmaceutical composition of the invention with the biological sample to form a mixture; and
c) detecting specifically bound antibody or bound fragment in the mixture which indicates the presence of GBS.

Alternatively, a method for the detection of antibody specific to a GBS antigen in a biological sample containing or suspected of containing said antibody may be performed as follows:
a) obtaining a biological sample from a host;
b) incubating a pharmaceutical composition of the invention with the biological sample to form a mixture; and
c) detecting specifically bound antigen or bound fragment in the mixture which indicates the presence of antibody specific to GBS.

One of skill in the art will recognize that this diagnostic test may take several forms, including an immunological test such as an enzyme-linked immunosorbent assay (ELISA), a radioimmunoassay or a latex agglutination assay, essentially to determine whether antibodies specific for the protein are present in an organism.

The DNA sequences encoding polypeptides of the invention may also be used to design DNA probes for use in detecting the presence of GBS in a biological sample suspected of containing such bacteria. The detection method of this invention comprises:
a) obtaining the biological sample from a host;
b) incubating one or more DNA probes having a DNA sequence encoding a polypeptide of the invention or fragments thereof with the biological sample to form a mixture; and
c) detecting specifically bound DNA probe in the mixture which indicates the presence of GBS bacteria.

The DNA probes of this invention may also be used for detecting circulating GBS i.e. GBS nucleic acids in a sample, for example using a polymerase chain reaction, as a method of diagnosing GBS infections. The probe may be synthesized using conventional techniques and may be immobilized on a solid phase, or may be labelled with a detectable label. A preferred DNA probe for this application is an oligomer having a sequence complementary to at least about 6 contiguous nucleotides of the GBS polypeptides of the invention. In a further embodiment, the preferred DNA probe will be an oligomer having a sequence complementary to at least about 15 contiguous nucleotides of the GBS polypeptides of the invention. In a further embodiment, the preferred DNA probe will be an oligomer having a sequence complementary to at least about 30 contiguous nucleotides of the GBS polypeptides of the invention. In a further embodiment, the preferred DNA probe will be an oligomer having a sequence complementary to at least about 50 contiguous nucleotides of the GBS polypeptides of the invention.

Another diagnostic method for the detection of GBS in a host comprises:
a) labelling an antibody reactive with a pharmaceutical composition of the invention with a detectable label;
b) administering the labelled antibody to the host; and
c) detecting specifically bound labelled antibody or labelled fragment in the host which indicates the presence of GBS.

A further aspect of the invention is the use of the pharmaceutical compositons of the invention as immunogens for the production of specific antibodies for the diagnosis and in particular the treatment of GBS infection. Suitable antibodies may be determined using appropriate screening methods, for example by measuring the ability of a particular antibody to passively protect against GBS infection in a test model. The antibody may be a whole antibody or an antigen-binding fragment thereof and may belong to any immunoglobulin class. The antibody or fragment may be of animal origin, specifically of mammalian origin and more specifically of murine, rat or human origin. It may be a natural antibody or a fragment thereof, or if desired, a recombinant antibody or antibody fragment. The term recombinant antibody or antibody fragment means antibody or antibody fragment which was produced using molecular biology techniques. The antibody or antibody fragments may be polyclonal, or preferably monoclonal. It may be specific for a number of epitopes associated with the GBS polypeptides but is preferably specific for one.

According to one aspect, the present invention provides the use of an antibody for prophylaxis and/or treatment of GBS infections.

A further aspect of the invention is the use of the antibodies directed to the pharmaceutical compositions of the invention for passive immunization. One could use the antibodies described in the present application.

A further aspect of the invention is a method for immunization, whereby an antibody raised by a pharmaceutical composition of the invention is administered to a host in an amount sufficient to provide a passive immunization.

In a further embodiment, the invention provides the use of a pharmaceutical composition of the invention in the manufacture of a medicament for the prophylactic or therapeutic treatment of GBS infection.

In a further embodiment, the invention provides a kit comprising a pharmaceutical composition of the invention for detection or diagnosis of GBS infection.

The present invention also provides purified and isolated Streptococcus polypeptides which may be used to prevent, diagnose and/or treat Streptococcus infection.

According to one aspect, the present invention provides an isolated polypeptide comprising a sequence chosen from SEQ ID NO: 2, 4, 6, 8, 10, 12 or fragments or analogs thereof.

According to one aspect, the present invention provides an isolated polypeptide having at least 70% identity to a second polypeptide comprising a sequence chosen from SEQ ID NO: 2, 4, 6, 8, 10, 12 or fragments or analogs thereof.

According to one aspect, the present invention provides an isolated polypeptide having at least 80% identity to a second polypeptide comprising a sequence chosen from SEQ ID NO: 2, 4, 6, 8, 10, 12 or fragments or analogs thereof.

According to one aspect, the present invention provides an isolated polypeptide having at least 90% identity to a second polypeptide comprising a sequence chosen from SEQ ID NO: 2, 4, 6, 8, 10, 12 or fragments or analogs thereof.

According to one aspect, the present invention provides an isolated polypeptide having at least 95% identity to a second polypeptide comprising a sequence chosen from SEQ ID NO: 2, 4, 6, 8, 10, 12 or fragments or analogs thereof.

According to one aspect, the present invention provides an isolated polypeptide having at least 98% identity to a second polypeptide comprising a sequence chosen from SEQ ID NO: 2, 4, 6, 8, 10, 12 or fragments or analogs thereof.

According to one aspect, the present invention provides an isolated polypeptide having at least 99% identity to a second polypeptide comprising a sequence chosen from SEQ ID NO: 2, 4, 6, 8, 10, 12 or fragments or analogs thereof.

According to one aspect, the present invention provides an isolated polypeptide having a sequence chosen from SEQ ID NO: 2, 4, 6, 8, 10, 12 or fragments or analogs thereof.

According to one aspect, the present invention provides an epitope bearing portion of a polypeptide comprising a sequence chosen from: SEQ ID NOs: 2, 4, 6, 8, 10, 12 or fragments or analogs or thereof.

According to one aspect, the present invention provides an isolated polypeptide comprising a sequence chosen from SEQ ID NO: 2, 4, 6, 8, 10 or 12.

According to one aspect, the present invention provides an isolated polypeptide having at least 70% identity to a second polypeptide comprising a sequence chosen from SEQ ID NO: 2, 4, 6, 8, 10 or 12.

According to one aspect, the present invention provides an isolated polypeptide having at least 80% identity to a second polypeptide comprising a sequence chosen from SEQ ID NO: 2, 4, 6, 8, 10 or 12.

According to one aspect, the present invention provides an isolated polypeptide having at least 90% identity to a second polypeptide comprising a sequence chosen from SEQ ID NO: 2, 4, 6, 8, 10 or 12.

According to one aspect, the present invention provides an isolated polypeptide having at least 95% identity to a second polypeptide comprising a sequence chosen from SEQ ID NO: 2, 4, 6, 8, 10 or 12.

According to one aspect, the present invention provides an isolated polypeptide having at least 98% identity to a second polypeptide comprising a sequence chosen from SEQ ID NO: 2, 4, 6, 8, 10 or 12.

According to one aspect, the present invention provides an isolated polypeptide having at least 99% identity to a second polypeptide comprising a sequence chosen from SEQ ID NO: 2, 4, 6, 8, 10 or 12.

According to one aspect, the present invention provides an isolated polypeptide having a sequence chosen from SEQ ID NO: 2, 4, 6, 8, 10 or 12.

According to one aspect, the present invention provides an epitope bearing portion of a polypeptide comprising a sequence chosen from: SEQ ID NOs: 2, 4, 6, 8, 10 or 12.

In a further embodiment, the polypeptides in accordance with the present invention are antigenic.

In a further embodiment, the polypeptides in accordance with the present invention are immunogenic.

In a further embodiment, the polypeptides in accordance with the present invention can elicit an immune response in a host.

In a further embodiment, the present invention also relates to polypeptides which are able to raise antibodies having binding specificity to the polypeptides of the present invention as defined above.

An antibody that “has binding specificity” is an antibody that recognizes and binds the selected polypeptide but which does not substantially recognize and bind other molecules in a sample, e.g., a biological sample. Specific binding can be measured using an ELISA assay in which the selected polypeptide is used as an antigen.

In an additional aspect of the invention there are provided antigenic/immunogenic fragments of the polypeptides of the invention, or of analogs thereof.

The fragments of the present invention should include one or more such epitopic regions or be sufficiently similar to such regions to retain their antigenic/immunogenic properties. Thus, for fragments according to the present invention the degree of identity is perhaps irrelevant, since they may be 100% identical to a particular part of a polypeptide or analog thereof as described herein. The present invention further provides fragments having at least 10 contiguous amino acid residues from the polypeptide sequences of the present invention. In one embodiment, at least 15 contiguous amino acid residues. In one embodiment, at least 20 contiguous amino acid residues.

The terms “fragment” or “variant,” when referring to a polypeptide of the invention, mean a polypeptide which retains substantially at least one of the biological functions or activities of the polypeptide. Such a biological function or activity can be, e.g., any of those described above, and includes having the ability to react with an antibody, i.e., having a epitope-bearing peptide. Fragments or variants of the polypeptides, e.g. of SEQ ID NOS: 2, 4, 6, 8, 10 or 12 have sufficient similarity to those polypeptides so that at least one activity of the native polypeptides is retained. Fragments or variants of smaller polypeptides, e.g., of the polypeptides of SEQ ID NOS: 2, 4, 6, 8, 10 or 12, retain at least one activity (e.g., an activity expressed by a functional domain thereof, or the ability to react with an antibody or antigen-binding fragment of the invention) of a comparable sequence found in the native polypeptide.

The key issue, once again, is that the fragment retains the antigenic/immunogenic properties.

The skilled person will appreciate that analogs of the polypeptides of the invention will also find use in the context of the present invention, i.e. as antigenic/immunogenic material. Thus, for instance proteins or polypeptides which include one or more additions, deletions, substitutions or the like are encompassed by the present invention.

As used herein, “fragments”, “analogs”, “variants” or “derivatives” of the polypeptides of the invention include those polypeptides in which one or more of the amino acid residues are substituted with a conserved or non-conserved amino acid residue (preferably conserved) and which may be natural or unnatural. In one embodiment, derivatives and analogs of polypeptides of the invention will have about 70% identity with those sequences illustrated in the figures or fragments thereof. That is, 70% of the residues are the same. In a further embodiment, polypeptides will have greater than 80% identity. In a further embodiment, polypeptides will have greater than 85% identity. In a further embodiment, polypeptides will have greater than 90% identity. In a further embodiment, polypeptides will have greater than 95% identity. In a further embodiment, polypeptides will have greater than 99% identity. In a further embodiment, analogs of polypeptides of the invention will have fewer than about 20 amino acid residue substitutions, modifications or deletions and more preferably less than 10.

A variant of a polypeptide of the invention may be, e.g., (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which additional amino acids are fused to the polypeptide, such as a leader or secretory sequence or a sequence which is employed for purification of the polypeptide, commonly for the purpose of creating a genetically engineered form of the protein that is susceptible to secretion from a cell, such as a transformed cell; The additional amino acids may be from a heterologous source, or may be endogenous to the natural gene.

Variant polypeptides belonging to type (i) above include, e.g., muteins, analogs and derivatives. A variant polypeptide can differ in amino acid sequence by, e.g., one or more additions, substitutions, deletions, insertions, inversions, fusions, and truncations or a combination of any of these. Variant polypeptides belonging to type (ii) above include, e.g., modified polypeptides. Known polypeptide modifications include, but are not limited to, glycosylation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formatin, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been-described in great detail in the scientific literature. Several particularly common modifications, glycosylation, lipid attachment, sulfation, gamma-carboxylation of glutamic acid residues, hydroxylation and ADP-ribosylation, for instance, are described in many basic texts, such as Proteins—Structure and Molecular Properties, 2nd ed., T. E. Creighton, W.H. Freeman and Company, New. York (1993). Many detailed reviews are available on this subject, such as by Wold, F., Posttranslationail Covalent Modification of Proteins, B. C. Johnson, Ed., Academic Press, New York 1-12 (1983); Seifter et al. (1990) Meth. Enzymol. 182:626-646 and Rattan et al. (1992) Ann. N.Y. Acad. Sci. 663:48-62.

Variant polypeptides belonging to type (iii) are well-known in the art and include, e.g., PEGulation or other chemical modifications.

Variants polypeptides belonging to type (iv) above include, e.g., preproteins or proproteins which can be activated by cleavage of the proprotein portion to produce an active mature polypeptide. Variants include a variety of hybrid, chimeric or fusion polypeptides. Typical examples of such variants are discussed elsewhere herein.

Many other types of variants are known to those of skill in the art. For example, as is well known; polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications or variations can occur anywhere in a polypeptide, including the peptide backbone, the amino acid side-chains and the amino or carboxyl termini. The same type of modification may be present in the same or varying degree at several sites in a given polypeptide. Also, a given polypeptide may contain more than one type of modification. Blockage of the amino or carboxyl group in a polypeptide, or both, by a covalent modification, is common in naturally-occurring and synthetic polypeptides. For instance, the aminoterminal residue of polypeptides made in E. coli, prior to proteolytic processing, is often N-formylmethionine. The modifications can be a function of how the protein is made. For recombinant polypeptides, for example, the modifications are determined by the host cell posttranslational modification capacity and the modification signals in the polypeptide amino acid sequence. Accordingly, when glycosylation is desired, a polypeptide can be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

Variant polypeptides can be fully functional or can lack function in one or more activities, e.g., in any of the functions or activities described above. Among the many types of useful variations are, e.g., those which exhibit alteration of catalytic activity. For example, one embodiment involves a variation at the binding site that results in binding but not hydrolysis, or slower hydrolysis, of cAMP. A further useful variation at the same site can result in altered affinity for cAMP. Useful variations also include changes that provide for affinity for another cyclic nucleotide. Another useful variation includes one that prevents activation by protein kinase A. Another useful variation provides a fusion protein in which one or more domains or subregions are operationally fused to one or more domains or subregions from another phosphodiesterase isoform or family.

In an alternative approach, the analogs could be fusion polypeptides, incorporating moieties which render purification easier, for example by effectively tagging the desired polypeptide. It may be necessary to remove the “tag” or it may be the case that the fusion polypeptide itself retains sufficient antigenicity to be useful.

In an alternative approach, the analogs or derivatives could be fusion polypeptides, incorporating moieties which render purification easier, for example by effectively tagging the desired protein or polypeptide, it may be necessary to remove the “tag” or it may be the case that the fusion polypeptide itself retains sufficient antigenicity to be useful.

In an additional aspect of the invention there are provided antigenic/immunogenic fragments of the proteins or polypeptides of the invention, or of analogs or derivatives thereof.

Thus, what is important for analogs, derivatives and fragments is that they possess at least a degree of the antigenicity/immunogenic of the protein or polypeptide from which they are derived.

Also included are polypeptides which have fused thereto other compounds which alter the polypeptides biological or pharmacological properties i.e. polyethylene glycol (PEG) to increase half-life; leader or secretory amino acid sequences for ease of purification; prepro- and pro-sequences; and (poly)saccharides.

Moreover, the polypeptides of the present invention can be modified by terminal —NH₂ acylation (eg. by acetylation, or thioglycolic acid amidation, terminal carboxy amidation, e.g. with ammonia or methylamine) to provide stability, increased hydrophobicity for linking or binding to a support or other molecule.

Also contemplated are hetero and homo polypeptide multimers of the polypeptide fragments and analogs. These polymeric forms include, for example, one or more polypeptides that have been cross-linked with cross-linkers such as avidin/biotin, gluteraldehyde or dimethylsuperimidate. Such polymeric forms also include polypeptides containing two or more tandem or inverted contiguous sequences, produced from multicistronic mRNAs generated by recombinant DNA technology. In a further embodiment, the present invention also relates to chimeric polypeptides which comprise one or more polypeptides or fragments or analogs thereof as defined in the figures of the present application.

In a further embodiment, the present invention also relates to chimeric polypeptides comprising two or more polypeptides having a sequence chosen from SEQ ID NOs: 2, 4, 6, 8, 10, 12 or fragments or analogs thereof; provided that the polypeptides are linked as to formed a chimeric polypeptide.

In a further embodiment, the present invention also relates to chimeric polypeptides comprising two or more polypeptides having a sequence chosen from SEQ ID NOs: 2, 4, 6, 8, 10 or 12 provided that the polypeptides are linked as to formed a chimeric polypeptide.

Preferably, a fragment, analog or derivative of a polypeptide of the invention will comprise at least one antigenic region i.e. at least one epitope.

In one embodiment, pharmaceutical compositions of the invention are used for the treatment or prophylaxis of Streptococcus infection and/or diseases and symptoms mediated by Streptococcus infection, in particular group B Streptococcus (GBS or S. agalactiae).

According to another aspect, there are provided polynucleotides encoding polypeptides characterized by the amino acid sequence comprising SEQ ID NO: 2, 4, 6, 8, 10, 12 or fragments or analogs thereof.

In one embodiment, polynucleotides are those illustrated in SEQ ID No: 1, 3, 5, 7, 9 or 11 which may include the open reading frames (ORF), encoding the polypeptides of the invention.

Those skilled in the art will appreciate that the invention includes DNA molecules, i.e. polynucleotides and their complementary sequences that encode analogs such as mutants, variants, homologues and derivatives of such polypeptides, as described herein in the present patent application. The invention also includes RNA molecules corresponding to the DNA molecules of the invention. In addition to the DNA and RNA molecules, the invention includes the corresponding polypeptides and monospecific antibodies that specifically bind to such polypeptides.

It will be appreciated that the polynucleotide sequences illustrated in the figures may be altered with degenerate codons yet still encode the polypeptides of the invention. Accordingly the present invention further provides polynucleotides which hybridize to the polynucleotide sequences herein above described (or the complement sequences thereof) having 70% identity between sequences. In one embodiment, at least 80% identity between sequences. In one embodiment, at least 85% identity between sequences. In one embodiment, at least 90% identity between sequences. In one embodiment, at least 95% identity. In a further embodiment, more than 97% identity.

In a further embodiment, polynucleotides are hybridizable under stringent conditions.

Suitable stringent conditions for hybridization can be readily determined by one of skilled in the art (see for example Sambrook et al., (1989) Molecular cloning: A Laboratory Manual, 2^{nd} ed, Cold Spring Harbor, N.Y.; Current Protocols in Molecular Biology, (1999) Edited by Ausubel F. M. et al., John Wiley & Sons, Inc., N.Y.).

“Suitable stringent conditions”, as used herein, means, for example, incubating a blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1×SSC and 0.1% SDS for 30 min at 65° C.), thereby selecting sequences having, e.g., 95% or greater sequence identity.

Other non-limiting examples of suitable stringent conditions include a final wash at 65° C. in aqueous buffer containing 30 mM NaCl and 0.5% SDS. Another example of suitable stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

In a further embodiment, the present invention provides polynucleotides that hybridize under stringent conditions to either
(a) a DNA sequence encoding a polypeptide or
(b) the complement of a DNA sequence encoding a polypeptide;
   wherein said polypeptide comprises SEQ ID NO: 2, 4, 6, 8, 10, 12 or fragments or analogs thereof.

In a further embodiment, the present invention provides polynucleotides that hybridize under stringent conditions to either
(a) a DNA sequence encoding a polypeptide or
(b) the complement of a DNA sequence encoding a polypeptide;
   wherein said polypeptide comprises SEQ ID NO: 2, 4, 6, 8, 10 or 12.

In a further embodiment, the present invention provides polynucleotides that hybridize under stringent conditions to either
(a) a DNA sequence encoding a polypeptide or
(b) the complement of a DNA sequence encoding a polypeptide;
   wherein said polypeptide comprises at least 10 contiguous amino acid residues from a polypeptide comprising SEQ ID NO: 2, 4, 6, 8, 10, 12 or fragments or analogs thereof.

In a further embodiment, the present invention provides polynucleotides that hybridize under stringent conditions to either
(a) a DNA sequence encoding a polypeptide or
(b) the complement of a DNA sequence encoding a polypeptide;
   wherein said polypeptide comprises at least 10 contiguous amino acid residues from a polypeptide comprising SEQ ID NO: 2, 4, 6, 8, 10 or 12.

In a further embodiment, polynucleotides are those encoding polypeptides of the invention illustrated in SEQ ID NO: 2, 4, 6, 8, 10, 12 or fragments or analogs thereof.

In a further embodiment, polynucleotides are those illustrated in SEQ ID NO: 1, 3, 5, 7, 9 or 11 encoding polypeptides of the invention or fragments or analogs thereof.

In a further embodiment, polynucleotides are those encoding polypeptides of the invention illustrated in SEQ ID NO: 2, 4, 6, 8, 10 or 12.

In a further embodiment, polynucleotides are those illustrated in SEQ ID NO: 1, 3, 5, 7, 9 or 11 encoding polypeptides of the invention.

As will be readily appreciated by one skilled in the art, polynucleotides include both DNA and RNA.

The present invention also includes polynucleotides complementary to the polynucleotides described in the present application.

A further aspect of the invention is the use of the antibodies directed to the polypeptides of the invention for passive immunization. One could use the antibodies described in the present application. Suitable antibodies may be determined using appropriate screening methods, for example by measuring the ability of a particular antibody to passively protect against streptococcal infection in a test model. One example of an animal model is the mouse model described in the examples herein. The antibody may be a whole antibody or an antigen-binding fragment thereof and may belong to any immunoglobulin class. The antibody or fragment may be of animal origin, specifically of mammalian origin and more specifically of murine, rat or human origin. It may be a natural antibody or a fragment thereof, or if desired, a recombinant antibody or antibody fragment. The term recombinant antibody or antibody fragment means antibody or antibody fragment which was produced using molecular biology techniques. The antibody or antibody fragments may be polyclonal, or preferably monoclonal. It may be specific for a number of epitopes associated with the Streptococcus polypeptides but is preferably specific for one.

In a further aspect, the invention provides a method for prophylactic or therapeutic treatment of streptococcal infection in a host susceptible to streptococcal infection comprising administering to the host a prophylactic or therapeutic amount of a pharmaceutical composition of the invention.

In a further embodiment, the invention provides the use of a pharmaceutical composition for the prophylactic or therapeutic treatment of streptococcal bacterial infection in a host susceptible to streptococcal infection comprising administering to said host a therapeutic or prophylactic amount of a composition of the invention.

In a further embodiment, the invention provides a kit comprising a polypeptide of the invention for detection or diagnosis of streptococcal infection.

Unless otherwise defined, all technical and scientific terms used herein have the same meaning as commonly understood by one of ordinary skill in the art to which this invention belongs. All publications, patent applications, patents and other references mentioned herein are incorporated by reference in their entirety. In case of conflict, the present specification, including definitions, will control. In addition, the materials, methods, and examples are illustrative only and not intended to be limiting.

### EXAMPLE 1

This example describes the production, purification, chemical coupling of the recombinant Sip (rSip) polypeptide to DPPE-MCC phospholipid and a procedure to generate liposome.

The sip gene from strain C388/90 (serotype Ia/c) was amplified from purified chromosomal DNA by PCR with recombinant Taq DNA Polymerase (Amersham Pharmacia Biotech) as described by the manufacturer. The following two primers OCRR-259 (5′-CCGGCAGATCTATGAAAATGAATAAAAAGGTACTATTG-31) (SEQ ID No:13) and OCRR-260 (5′-GGCCGTCTAGATTATTTGTTAAATGATACGTGAACA-3′) (SEQ ID No:14) which respectively contained the BglII and XbaI restriction sites were used to perform the amplifications. PCR was performed with 10 cycles of 30 sec at 94° C., 20 sec at 56° C. and 30 sec at 72° C., followed by 25 cycles of 30 sec at 94° C., 20 sec at 70° C. and 30 sec at 72° C. and a final elongation period of 5 min at 72° C. The amplification product was ligated into plasmid pURV22 and after sequencing, the recombinant plasmid named pURV32 was transformed into E. coli XL1-Blue MRF′.

The purified recombinant plasmid pURV32 was used to transform E. coli strain BLR (F⁻ ompT hSdS_{B}(r⁻_{B}m⁻_{B}) gal dcm Δ(srl-recA)306::Tn10 (Tc^{R}) (Novagen) by electroporation (Gene Pulser II apparatus, BIO-RAD Labs, Mississauga, Ontario, Canada). This recombinant strain was inoculated in LB broth (Gibco BRL) containing 40 μg/ml of kanamycin, and was first incubated at 35° C. for approximately 2 h with agitation (OD₆₀₀ₙₘ=0.6) after which time the temperature was increased to 40° C. for an additional 3 h in order to induce the production of the recombinant polypeptide. After the induction period, it was observed that rSip polypeptide could be found inside the bacterial cells as well as in the culture supernatant. The rSip polypeptide was purified from the culture supernatant as described in the following lines. The bacterial cells were removed from the culture media by centrifugation at 15,000×g for 10 min at 4° C. The supernatant was then filtered onto a 0.45 μm membrane and 20 mM Tris base, 5 mM ethylenediaminetetraacetic acid (EDTA), 2 mM dithiothreitol (DTT), and 1M ammonium sulfate were added. The rSip polypeptide was purified from the other molecules present in the culture supernatant by three successive chromatographic steps; first by hydrophobic interaction chromatography using Phenyl Sepharose FF (Amersham Pharmacia Biotech), followed by an anionic exchange chromatography using Q-Speharose HP resin (Amersham Pharmacia Biotech), and an hydrophobic interaction chromatography using a Butyl Sepharose FF (Amersham Pharmacia Biotech). The purity of rSip polypeptide was evaluated to be approximately 95% by SDS-PAGE and the amount of polypeptide was determined by the MicroBCA according to the manufacturer's instructions (Pierce Chemical Company, Rockford, Ill.).

Chemical coupling of purified rSip to liposome via the DPPE-MCC phospholipid (Dipalmitoyl-sn-glycero-3-phosphoethanolamine-N-(4-(p-maleimidomethyl)cyclohexane-carboxamine)(Avanti polar lipids, Alabaster, Ala.) was performed following the method described by Harokopakis et al. 1995 (J. Immunol. Methods, 185; 31-42). Briefly, liposomes made of Dipalmitoyl-sn-Glycerol-3-phosphocholine (DPPC; Avanti), DC-cholesterol (DC-Chol; Avanti), and DPPE-MCC were prepared by rapid dilution. To generate the bilayer membrane liposomes, the lipids were dissolved in a chloroform:methanol solution (2:1) in a round bottom glass flask and dried using a rotatory evaporator (Rotavapor, Buchi, Switzerland) to achieve an even film on the vessel. This lipid film was resuspended with a phosphate buffered solution (137 mM NaCl, 1 mM KH2PO4, 9 mM Na2PO4, 2.7 mM KCl, pH 7.4) at 56° C. The solution was then kept at room temperature for 1 h with agitation. The resulting milky solution was sequentially extruded through 1000, 400, 200, and 100 nm polycarbonate filters using a stainless steel extrusion device (Lipex Biomembranes, Vancouver, Canada). Lipid aggregates were removed by centrifugation at 20000×g for 15 min at 4° C. The liposome solution was centrifuged at 200000×g for 1 h at 4° C. and the pellet was suspended in PBS buffer. Vesicle size and homogeneity were evaluated by quasi-elastic light scattering with a submicron particles analyzer (model N4 Plus, Beckman Coulter). Using this apparatus, it was estimated that the liposome size was approximately 100 nm.

To couple rSip to the DPPE-MCC-containing liposomes, thiol groups were added to free amino groups of the rSip by means of the amine-reactive reagent SPDP (molar ratio 22:1) (N-succinimidyl 3-(2-pyridyldithio) propionate; Pierce), resulting in rSip-thiopropionate (rSip-TP). The thiol groups are necessary for reaction with the maleimide group of the DPPE-MCC constituent of the liposomes. rSip-TP was then reduced using DTT, and excess free pyridine-2-thione, SPDP, and DTT were removed by gel filtration using a P-6 desalting column. The reduced rSip polypeptide was incubated under nitrogen atmosphere at a final concentration of 1 mg/ml with the liposome suspension. The resulting rSip-liposomes were separated from free polypeptide by ultracentrifugation (200000×g, 1 h at room temperature) and resuspended in an equal volume of PBS buffer containing 0.3M sucrose. Liposome preparations were sterilized by filtration through a 0.22 μm membrane and stored at −80° C. until used. The amount of rSip-liposome was evaluated by MicroBCA (Pierce) after solubilization of rSip-liposome preparations with chloroform:methanol solution (2:1).

To better control the chemical coupling of rSip to the DPPE-MCC phospholipids, an alternate strategy which is described in Example 2 was also evaluated.

### EXAMPLE 2

This example describes the cloning strategy used to generate C1rSip and C2rSip, the production of these recombinant polypeptides in E. coli, and their purification.

In order to increase the efficiency of the coupling reaction and to generate Sip-liposomes that are more stable, modified rSip polypeptides where at least one cysteine residue was added to the amino acid sequence have been designed. Preferably, these cysteine residues are added at positions where it does not remove the antigenic properties of the original Sip polypeptide.

Sip polypeptides of the invention include Sip polypeptides to which at least one cysteine has been added in order to be coupled to the liposome.

The modifications to the original sip gene sequence are presented in Table 1. To generate the polypeptides C1rSip and C2rSip, mutagenesis experiments using the Quickchange Site-Directed Mutagenesis kit from Stratagene, the oligonucleotides described in Table 2, and the sip genes cloned in the pURV22 vector (Example 1) were performed according to the manufacturer's recommendations.

Each of the resultant plasmid constructs was used to transform by electroporation (Gene Pulser II apparatus, BIO-RAD Labs, Mississauga, Ontario, Canada) E. coli strain BLR (F⁻ ompT hsdS_{B}(r⁻_{B}m⁻_{B}) gal dcm Δ(srl-recA)306::Tn10 (Tc^{R}) (Novagen). This recombinant strain was inoculated in LB broth (Gibco BRL) containing 40 μg/ml of kanamycin, and was first incubated at 35° C. for approximately 2 h with agitation (OD₆₀₀ₙₘ=0.6) after which time the temperature was increased to 40° C. for an additional 3 h in order to induce the production of the recombinant polypeptide. As for rSip polypeptide, The C1rSip and C2rSip polypeptides were also purified from culture supernatant as described in Example 1.

TABLE 1
List of the modified sip genes compared to the SEQ
ID No: 1.

| Gene/Polypeptide | |
|---|---|
| designation | Characteristics |
| C1rSip | Codon TGT was added between the nucleic acids |
| | 1302 and 1303 on SEQ ID No: 1 to become SEQ ID |
| | No: 3 |
| C2rSip | A exchange for T (position 238) |
| | A exchange for G (position 239) |
| | The codon TGT added between the nucleic acids |
| | 1302 and 1303 on SEQ ID No: 1 to become SEQ ID |
| | No: 5 |

TABLE 2
List of PCR oligonucleotide primer pairs designed
for the generation of modified Sip genes listed in Table 1.

| Gene/Polypeptide | PCR- | Sequence 5′-3′ |
|---|---|---|
| designation | primer ID | (SEQ ID No:) |
| C1rSip | DMAR2702 | CACGTATCATTTAACAAATGTTAATCTAGAGCTTGAGGATCCGGC |
| | | (SEQ ID No: 15) |
| | DMAR2703 | GCCGGATCCTCAAGCTCTAGATTAACATTTGTTAAATGATACGTG |
| | | (SEQ ID No: 16) |
| C2rSip | DMAR2483 | GAATGTCTTAGCAAAAATTAATTGCATTGCAGATATCAATC |
| | | (SEQ ID No: 17) |
| | DMAR2484 | GATTGATATCTGCAATGCAATTAATTTTTGCTAAGACATTC |
| | | (SEQ ID No: 18) |
| | DMAR2702 | CACGTATCATTTAACAAATGTTAATCTAGAGCTTGAGGATCCGGC |
| | | (SEQ ID No: 19) |
| | DMAR2703 | GCCGGATCCTCAAGCTCTAGATTAACATTTGTTAAATGATACGTG |
| | | (SEQ ID No: 20) |

### EXAMPLE 3

This example illustrates the coupling of C1rSip and C2rSip to DPPE-MCC phospholipid.

In order to couple the C1rSip and C2rSip to DPPE-MCC phospholipid, liposomes containing DPPE-MCC lipid were prepared as described in Example 1. Before coupling, the C1rSip and C2rSip polypeptides were treated with 25 mM DTT during 30 min at room temperature. After the incubation, DTT was removed by gel filtration using a P-6 desalting column. The treated-C1rSip and -C2rSip polypeptides were incubated under nitrogen atmosphere at a final concentration of 2 mg/ml with the liposome suspension containing 0.01% Triton X-100. The resulting C1rSip-, C2rSip-liposomes were separated from free protein by ultracentrifugation (200000×g for 1 h at room temperature) and resuspended in an equal volume of PBS buffer containing 0.3M sucrose. Liposome preparations were sterilized by filtration through a 0.22 μm membrane and stored at −80° C. until used. The amount of C1rSip- and C2rSip-bound to the liposome was evaluated by MicroBCA (Pierce) after solubilization of C1rSip-, and C2rSip-liposome with chloroform:methanol solution (2:1).

In order to evaluate the stability of these liposome preparations, samples from these preparations that were kept at 4° C. and 37° C. for a period of 8 weeks were analyzed by SDS-PAGE gels and by quasi-elastic light scattering with a submicron particles analyzer. Overtime, some polypeptide degradation and vesicle size increases resulting from aggregation were observed for rSip-SPDP-liposome formulation (Table 3). No such polypeptide degradation nor aggregation were recorded for different lots of C1rSip- and C2rSip-liposome. This result suggested that the C1rSip- and C2rSip-liposome formulations might be more stable and homogeneous than the rSip-SPDP-liposome formulation

Table 3. Stability of liposome formulations, determination of anti-Sip titer (μg/ml) for sera collected from immunized mice and ability of Sip-liposome formulations to elicit protection against GBS strain C388/90 (I a/c)

| | | Anti-rSip | Protection: Nb |
|---|---|---|---|
| | Stability and | titer post- | of surviving |
| Antigen | aggregation with | 3^{rd} dose | mice/total mice |
| description¹ | time² | (μg/ml) | (% survival) |
| rSip-SPDP- | S1; A++ | 1209 ± 622 | 58/71 (82) |
| liposome | | | |
| C1rSip- | S3; A− | 1237 ± 412 | 48/54 (89) |
| liposome | | | |
| C2rSip- | S2; A− | 1492 ± 655 | 66/72 (92) |
| liposome | | | |
| rSip/AlOH | ND | 2883 ± 1592 | 9/9 (100) |
| PBS | ND | 2 ± 1 | 1/9 (11) |

¹Liposome description: DPPC:DPPE-MCC:DC-CHOL (4.5:0.5:5)
²S1/S3: Evaluation of the stability of the liposome formulations: 3 indicates very stable formulation, where no polypeptide degradation was observed, while 1 indicates that some polypeptide degradation was observed. A++ indicates that aggregation of the liposome vesicles was observed during the stability monitoring period; A− indicates that no aggregation was recorded for the formulation. ND; not done.

### EXAMPLE 4

This example describes the immunization of CD-1 mice with rSip/C1rSip/C2rSip-liposome formulations.

Groups of female CD-1 mice (Charles River Laboratories, St-Constant, Quebec, Canada) were immunized intramuscularly (IM) three times at two-week intervals with various amounts (0.001 to 20 μg) of purified rSip-, C1rSip- or C2rSip-liposome formulations. Group of mice were also immunized with 20 μg of purified rSip adsorbed to 10% aluminium hydroxide adjuvant (AlOH; Alhydrogel™ 2%: Brenntag Biosector, Denmark) or PBS containing 10% AlOH as control. For the dose-response evaluation, various volume of polypeptide-free liposome were added to certain formulations to maintain the total amount of phospholipids and DC-cholesterol constant from one formulation to the others. Blood samples were collected from the orbital sinus prior to each immunization and two weeks after the last injection. The serum samples were stored at −20° C.

### EXAMPLE 5

This example describes the analysis by ELISA of mouse sera following immunization with different liposome formulations.

Determination of Sip-specific antibody titers in the sera collected from CD-1 mice were determined by ELISA. Briefly, 100 μL of carbonate buffer (15 mM Na₂CO₃, 35 mM NaHCO₃ [pH 9.6]) containing purified rSip at a concentration of 1 μg/mL were added to each well of a flat-bottom microtitration plate (Falcon 3415, Becton Dickinson, Franklin Lakes, N.J.) and incubated overnight at RT. The plate was washed three times with PBS containing 0.05% (vol/vol) Tween 20. The mouse sera were serially diluted in PBS-Tween buffer and 100 μL of each dilution were added to the appropriate well. The plate was incubated for 90 minutes at RT and then washed three times with PBS-Tween buffer. One hundred μL horseradish-peroxidase-conjugated goat-anti-mouse immunoglobulin G (KPL: Kikegaard-Perry Laboratories, Gaithersburg, Md.) diluted in PBS-Tween buffer were added to each well, and the plate was incubated for 60 min at RT. The plates were washed three times and 100 μL of tetramethylbenzidine substrate (KPL) were added and incubated 10 min at RT. The reaction was stopped by the addition of 100 μl of 1M phosphoric acid. The OD₄₅₀ₙₘ was read with a SpectraMax 340 (Molecular Devices Corporation, Sunnyvale, Calif.) microplate reader. Mouse antibody concentration was calculated by interpolation on calibration curves generated for each plate, using wells coated with anti-Ig antibodies (KPL) in duplicate and known concentrations of purified mouse immunoglobulins (Sigma-Aldrich).

The three Sip-based liposome formulations tested (rSip-SPDP-liposome; C1rSip-liposome and C2rSip-liposome) induced post-dose-3 comparable amounts of Sip-specific antibodies in mice as evaluated by ELISA (Table 3). As presented in Table 4, the concentration of Sip-specific antibodies correlated with the amount of C1rSip-liposome injected to the animals. This latter result indicates that the amount of antigen injected to the animal influence the magnitude of the specific immune response. Specific-Sip antibodies were even detected in sera of mice injected with very low amounts of C1rSip-liposome, such as 1 ng, indicating that this formulation is highly efficient at inducing an humoral immune response.

### EXAMPLE 6

This example illustrates the protection of mice against fatal Group B streptococcal infection induced by immunization with different liposome-based formulations.

Three weeks following the third immunization, mice were challenged with approximately 3×10⁵ CFU of the Group B streptococcal strain C388/90 (Ia/c). Samples of the Group B streptococcal challenge inoculum were plated on blood agar plates to determine the CFU and to verify the challenge dose. Deaths were recorded for a period of 7 days. Survival rates higher than 82% were recorded in the groups of the mice injected with either three Sip-liposome formulations (Table 3). Survival rates below 18% were recorded for the control mice injected with 10% AlOH. The dose-reponse study presented in Table 4 indicated that three injections of only 5 ng of C1rSip-liposome efficiently protected CD-1 mice against a deadly GBS challenge, while only 2 out of the 9 mice injected with 1 ng survived. To maintain the amount of phospholipids and DC-cholesterol constant, polypeptide-free liposome were added to several formulations. To evaluate the impact of lower concentrations of lipids on the development of the specific immune response, polypeptide-free liposomes were not added to two formulations containing 25 and 50 ng of C1rSip (Table 4). Post-dose-3 ELISA results indicated that the level of Sip-specific antibodies induced by 50 ng of C1rSip-liposome with the addition of polypeptide free-liposome (671±300) is comparable to the level (497±228) of antibodies induced by 50 ng of C1rSip-liposome without the addition of polypeptide free-liposome. The survival rates recorded for mice injected with 25 and 50 ng with and without the addition of polypeptide free-liposome were also similar. These data suggest that the presence of small amounts of lipids and DC-cholesterol with C1rSip polypeptide are sufficient to induce a protective immune response.

TABLE 4
Evaluation of the protection conferred to mice by
increasing amounts of C1rSip-liposome formulations

| | | | Protection: Nb |
|---|---|---|---|
| Antigen Description | | | of surviving |
| | Volume of | Anti-rSip | mice/total |
| μg of C1rSip- | polypeptide- | titer post-3^{rd} | mice |
| liposome | free liposome | dose (μg/ml) | (% survival) |
| 20 | 0 | 2372 ± 715 | 4/9 (44) |
| 10 | 10 | 2811 ± 757 | 8/9 (89) |
| 5 | 15 | 2532 ± 496 | 9/9 (100) |
| 1 | 19 | 1694 ± 346 | 16/18 (89) |
| 0.5 | 19.5 | 1267 ± 400 | 9/9 (100) |
| 0.1 | 19.9 | 975 ± 467 | 7/9 (78) |
| 0.05 | 19.95 | 671 ± 300 | 16/18 (89) |
| 0.05 | 0 | 497 ± 228 | 9/9 (100) |
| 0.025 | 19.975 | ND | 9/9 (100) |
| 0.025 | 0 | ND | 9/9 (100) |
| 0.01 | 19.99 | 246 ± 161 | 14/18 (78) |
| 0.005 | 19.995 | 83 ± 54 | 15/18 (83) |
| 0.001 | 19.999 | 19 ± 15 | 2/9 (22) |
| 20 rSip-10% | 0 | 2423 ± 870 | 24/26 (92) |
| AlOH | | | |
| PBS-10% AlOH | 0 | 0 ± 0 | 5/27 (18) |

We claim:

1. A pharmaceutical composition comprising a liposome associated with an isolated polypeptide chosen from:
a) a polypeptide having at least 70% identity to a second polypeptide comprising SEQ ID No: 2, 4, 6, 8, 10, 12 or fragments or analogs thereof;
b) a polypeptide having at least 80% identity to a second polypeptide comprising SEQ ID No: 2, 4, 6, 8, 10, 12 or fragments or analogs thereof;
c) a polypeptide having at least 95% identity to a second polypeptide comprising SEQ ID No: 2, 4, 6, 8, 10, 12 or fragments or analogs thereof;
d) a polypeptide having at least 98% identity to a second polypeptide comprising SEQ ID No: 2, 4, 6, 8, 10, 12 or fragments or analogs thereof;
e) a polypeptide having at least 99% identity to a second polypeptide comprising SEQ ID No: 2, 4, 6, 8, 10, 12 or fragments or analogs thereof;
f) a polypeptide comprising SEQ ID No: 2, 4, 6, 8, 10, 12 or fragments or analogs thereof;
g) a polypeptide capable of raising antibodies having binding specificity for a polypeptide comprising SEQ ID No: 2, 4, 6, 8, 10, 12 or fragments or analogs thereof;
h) an epitope bearing portion of a polypeptide comprising SEQ ID No: 2, 4, 6, 8, 10, 12 or fragments or analogs thereof;
i) the polypeptide of (a), (b), (c), (d), (e), (f), (g) or (h) wherein the N-terminal Met residue is deleted;
the polypeptide of (a), (b), (c), (d), (e), (f), (g), (h) or (i) wherein the secretory amino acid sequence is deleted.

2. A pharmaceutical composition of claim 1, comprising at least one lipid selected from:
1,2-Dilauroyl-sn-Glycero-3-Phosphate (DLPA),
Dimyristoyl-sn-Glycero-3-Phosphate (DMPA),
1,2-Dipalmitoyl-sn-Glycero-3-Phosphate (DPPA),
1,2-Distearoyl-sn-Glycero-3-Phosphate (DSPA),
1,2-Dioleoyl-sn-Glycero-3-Phosphate (DOPA),
1-Palmitoyl-2-Oleoyl-sn-Glycero-3-Phosphate (POPA),
1,2-Dilauroyl-sn-Glycero-3-Phosphocholine (DLPC),
1,2-Ditridecanoyl-sn-Glycero-3-Phosphocholine,
1,2-Dimyristoyl-sn-Glycero-3-Phosphocholine (DMPC),
1,2-Dipentadecanoyl-sn-Glycero-3-Phosphocholine,
1,2-Dipalmitoyl-sn-Glycero-3-Phosphocholine (DPPC),
1,2-Diheptadecanoyl-sn-Glycero-3-Phosphocholine,
1,2-Distearoyl-sn-Glycero-3-Phosphocholine (DSPC),
1,2-Dimyristoleoyl-sn-Glycero-3-Phosphocholine,
1,2-Dipalmitoleoyl-sn-Glycero-3-Phosphocholine,
1,2-Dioleoyl-sn-Glycero-3-Phosphocholine (DOPC),
1-Myristoyl-2-Palmitoyl-sn-Glycero-3-Phosphocholine,
1-Myristoyl-2-Stearoyl-sn-Glycero-3-Phosphocholine,
1-Palmitoyl-2-Myristoyl-sn-Glycero-3-Phosphocholine,
1-Palmitoyl-2-Stearoyl-sn-Glycero-3-Phosphocholine,
1-Palmitoyl-2-Oleoyl-sn-Glycero-3-Phosphocholine (POPC),
1-Palmitoyl-2-Linoleoyl-sn-Glycero-3-Phosphocholine,
1,2-Dilauroyl-sn-Glycero-3-Phosphoethanolamine (DLPE),
1,2-Dimyristoyl-sn-Glycero-3-Phosphoethanolamine (DMPE),
1,2-Dipalmitoyl-sn-Glycero-3-Phosphoethanolamine (DPPE),
1,2-Dipalmitoleoyl-sn-Glycero-3-Phosphoethanolamine,
1,2-Distearoyl-sn-Glycero-3-Phosphoethanolamine (DSPE),
1,2-Dioleoyl-sn-Glycero-3-Phosphoethanolamine (DOPE),
1-Palmitoyl-2-Oleoyl-sn-Glycero-3-Phosphoethanolamine (POPE),
1,2-Dilauroyl-sn-Glycero-3-[Phospho-RAC-(1-glycerol)] (DLPG),
1,2-Dimyristoyl-sn-Glycero-3-[Phospho-RAC-(1-glycerol)] (DMPG),
1,2-Dipalmitoyl-sn-Glycero-3-[Phospho-RAC-(1-glycerol)] (DPPG),
1,2-Distearoyl-sn-Glycero-3-[Phospho-RAC-(1-glycerol)] (DSPG),
1,2-Dioleoyl-sn-Glycero-3-[Phospho-RAC-(1-glycerol)] (DOPG),
1-Palmitoyl-2-Oleoyl-sn-Glycero-3-[Phospho-RAC-(1-glycerol)] (POPG),
1,2-Dilauroyl-sn-Glycero-3-[Phospho-L-Serine] (DLPS),
1,2-Dimyristoyl-sn-Glycero-3-[Phospho-L-Serine] (DMPS),
1,2-Dipalmitoyl-sn-Glycero-3-[Phospho-L-Serine] (DPPS),
1,2-Distearoyl-sn-Glycero-3-[Phospho-L-Serine] (DSPS),
1,2-Dioleoyl-sn-Glycero-3-[Phospho-L-Serine] (DOPS),
1-Palmitoyl-2-Oleoyl-sn-Glycero-3-[Phospho-L-Serine] (POPS),
1,2-Dioleoyl-3-Trimethylammonium-Propane (DOTAP),
1,2-Dipalmitoyl-3-Trimethylammonium-Propane (DPTAP),
1,2-Distearoyl-3-Trimethylammonium-Propane (DSTAP), or
1,2-Dimyristoyl-3-Trimethylammonium-Propane (DMTAP).

3. A pharmaceutical composition of claim 1, wherein said polypeptide is coupled to DPPE-MCC phospholipid.

4. A pharmaceutical composition of claim 1, wherein said liposome comprises DPPC:DPPE-MCCE:DC-CHOL.

5. A pharmaceutical composition of claim 4, wherein said lipids are in the ratio of 4.5:0.5:5.

6. A method for the prophylatic oe therapeutic treatment of GBS infection, comprising:
administering an effective amount of a composition of claim 1.

7. A method for the prophylatic or therapeutic treatment of GBS infection, comprising:
administering an effective amount of a composition of claim 2.

8. A method for the prophylatic or therapeutic treatment of GBS infection, comprising:
administering an effective amount of a composition of claim 3.

9. A method for the prophylatic or therapeutic treatment of GBS infection, comprising:
administering an effective amount of a composition of claim 4.

10. A method for the prophylatic or therapeutic treatment of GBS infection, comprising:
administering an effective amount of a composition of claim 5.

11. A method of claim 1, wherein said GBS is Streptococcus.

12. A method of claim 2, wherein said GBS is Streptococcus.

13. A method of claim 3, wherein said GBS is Streptococcus.

14. A method of claim 4, wherein said GBS is Streptococcus.


SEQUENCE LISTING (20 sequences)

SEQ ID NO: 1 - DNA, 1305 bp, Streptococcus agalactiae
atgaaaatgaataaaaaggtactattgacatcgacaatggcagcttcgctattatcagtcgcaagtgttcaagcacaagaaacagatacgacgtggacagcacgtactgtttcagaggtaaaggctgatttggtaaagcaagacaataaatcatcatatactgtgaaatatggtgatacactaagcgttatttcagaagcaatgtcaattgatatgaatgtcttagcaaaaattaataacattgcagatatcaatcttatttatcctgagacaacactgacagtaacttacgatcagaagagtcatactgccacttcaatgaaaatagaaacaccagcaacaaatgctgctggtcaaacaacagctactgtggatttgaaaaccaatcaagtttctgttgcagaccaaaaagtttctctcaatacaatttcggaaggtatgacaccagaagcagcaacaacgattgtttcgccaatgaagacatattcttctgcgccagctttgaaatcaaaagaagtattagcacaagagcaagctgttagtcaagcagcagctaatgaacaggtatcaacagctcctgtgaagtcgattacttcagaagttccagcagctaaagaggaagttaaaccaactcagacgtcagtcagtcagtcaacaacagtatcaccagcttctgttgccgctgaaacaccagctccagtagctaaagtagcaccggtaagaactgtagcagcccctagagtggcaagtgttaaagtagtcactcctaaagtagaaactggtgcatcaccagagcatgtatcagctccagcagttcctgtgactacgacttcaacagctacagacagtaagttacaagcgactgaagttaagagcgttccggtagcacaaaaagctccaacagcaacaccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcaggtgatccaggtgatcatggtaaaggtttagcagtcgactttattgtaggtaaaaaccaagcacttggtaatgaagttgcacagtactctacacaaaatatggcagcaaataacatttcatatgttatctggcaacaaaagttttactcaaatacaaatagtatttatggacctgctaatacttggaatgcaatgccagatcgtggtggcgttactgccaaccattatgaccatgttcacgtatcatttaacaaataa

SEQ ID NO: 2 - protein, 434 aa, Streptococcus agalactiae
MKMNKKVLLTSTMAASLLSVASVQAQETDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVSVADQKVSLNTISEGMTPEAATTIVSPMKTYSSAPALKSKEVLAQEQAVSQAAANEQVSTAPVKSITSEVPAAKEEVKPTQTSVSQSTTVSPASVAAETPAPVAKVAPVRTVAAPRVASVKVVTPKVETGASPEHVSAPAVPVTTTSTATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNK

SEQ ID NO: 3 - DNA, 1308 bp, Streptococcus agalactiae
atgaaaatgaataaaaaggtactattgacatcgacaatggcagcttcgctattatcagtcgcaagtgttcaagcacaagaaacagatacgacgtggacagcacgtactgtttcagaggtaaaggctgatttggtaaagcaagacaataaatcatcatatactgtgaaatatggtgatacactaagcgttatttcagaagcaatgtcaattgatatgaatgtcttagcaaaaattaataacattgcagatatcaatcttatttatcctgagacaacactgacagtaacttacgatcagaagagtcatactgccacttcaatgaaaatagaaacaccagcaacaaatgctgctggtcaaacaacagctactgtggatttgaaaaccaatcaagtttctgttgcagaccaaaaagtttctctcaatacaatttcggaaggtatgacaccagaagcagcaacaacgattgtttcgccaatgaagacatattcttctgcgccagctttgaaatcaaaagaagtattagcacaagagcaagctgttagtcaagcagcagctaatgaacaggtatcaacagctcctgtgaagtcgattacttcagaagttccagcagctaaagaggaagttaaaccaactcagacgtcagtcagtcagtcaacaacagtatcaccagcttctgttgccgctgaaacaccagctccagtagctaaagtagcaccggtaagaactgtagcagcccctagagtggcaagtgttaaagtagtcactcctaaagtagaaactggtgcatcaccagagcatgtatcagctccagcagttcctgtgactacgacttcaacagctacagacagtaagttacaagcgactgaagttaagagcgttccggtagcacaaaaagctccaacagcaacaccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcaggtgatccaggtgatcatggtaaaggtttagcagtcgactttattgtaggtaaaaaccaagcacttggtaatgaagttgcacagtactctacacaaaatatggcagcaaataacatttcatatgttatctggcaacaaaagttttactcaaatacaaatagtatttatggacctgctaatacttggaatgcaatgccagatcgtggtggcgttactgccaaccattatgaccatgttcacgtatcatttaacaaatgttaa

SEQ ID NO: 4 - protein, 435 aa, Streptococcus agalactiae
MKMNKKVLLTSTMAASLLSVASVQAQETDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVSVADQKVSLNTISEGMTPEAATTIVSPMKTYSSAPALKSKEVLAQEQAVSQAAANEQVSTAPVKSITSEVPAAKEEVKPTQTSVSQSTTVSPASVAAETPAPVAKVAPVRTVAAPRVASVKVVTPKVETGASPEHVSAPAVPVTTTSTATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNKC

SEQ ID NO: 5 - DNA, 1308 bp, Streptococcus agalactiae
atgaaaatgaataaaaaggtactattgacatcgacaatggcagcttcgctattatcagtcgcaagtgttcaagcacaagaaacagatacgacgtggacagcacgtactgtttcagaggtaaaggctgatttggtaaagcaagacaataaatcatcatatactgtgaaatatggtgatacactaagcgttatttcagaagcaatgtcaattgatatgaatgtcttagcaaaaattaattgcattgcagatatcaatcttatttatcctgagacaacactgacagtaacttacgatcagaagagtcatactgccacttcaatgaaaatagaaacaccagcaacaaatgctgctggtcaaacaacagctactgtggatttgaaaaccaatcaagtttctgttgcagaccaaaaagtttctctcaatacaatttcggaaggtatgacaccagaagcagcaacaacgattgtttcgccaatgaagacatattcttctgcgccagctttgaaatcaaaagaagtattagcacaagagcaagctgttagtcaagcagcagctaatgaacaggtatcaacagctcctgtgaagtcgattacttcagaagttccagcagctaaagaggaagttaaaccaactcagacgtcagtcagtcagtcaacaacagtatcaccagcttctgttgccgctgaaacaccagctccagtagctaaagtagcaccggtaagaactgtagcagcccctagagtggcaagtgttaaagtagtcactcctaaagtagaaactggtgcatcaccagagcatgtatcagctccagcagttcctgtgactacgacttcaacagctacagacagtaagttacaagcgactgaagttaagagcgttccggtagcacaaaaagctccaacagcaacaccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcaggtgatccaggtgatcatggtaaaggtttagcagtcgactttattgtaggtaaaaaccaagcacttggtaatgaagttgcacagtactctacacaaaatatggcagcaaataacatttcatatgttatctggcaacaaaagttttactcaaatacaaatagtatttatggacctgctaatacttggaatgcaatgccagatcgtggtggcgttactgccaaccattatgaccatgttcacgtatcatttaacaaatgttaa

SEQ ID NO: 6 - protein, 435 aa, Streptococcus agalactiae
MKMNKKVLLTSTMAASLLSVASVQAQETDTTWTARTVSEVKADLVKQDNKSSYTVKYGDTLSVISEAMSIDMNVLAKINCIADINLIYPETTLTVTYDQKSHTATSMKIETPATNAAGQTTATVDLKTNQVSVADQKVSLNTISEGMTPEAATTIVSPMKTYSSAPALKSKEVLAQEQAVSQAAANEQVSTAPVKSITSEVPAAKEEVKPTQTSVSQSTTVSPASVAAETPAPVAKVAPVRTVAAPRVASVKVVTPKVETGASPEHVSAPAVPVTTTSTATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNKC

SEQ ID NO: 7 - DNA, 1230 bp, Streptococcus agalactiae
caagaaacagatacgacgtggacagcacgtactgtttcagaggtaaaggctgatttggtaaagcaagacaataaatcatcatatactgtgaaatatggtgatacactaagcgttatttcagaagcaatgtcaattgatatgaatgtcttagcaaaaattaataacattgcagatatcaatcttatttatcctgagacaacactgacagtaacttacgatcagaagagtcatactgccacttcaatgaaaatagaaacaccagcaacaaatgctgctggtcaaacaacagctactgtggatttgaaaaccaatcaagtttctgttgcagaccaaaaagtttctctcaatacaatttcggaaggtatgacaccagaagcagcaacaacgattgtttcgccaatgaagacatattcttctgcgccagctttgaaatcaaaagaagtattagcacaagagcaagctgttagtcaagcagcagctaatgaacaggtatcaacagctcctgtgaagtcgattacttcagaagttccagcagctaaagaggaagttaaaccaactcagacgtcagtcagtcagtcaacaacagtatcaccagcttctgttgccgctgaaacaccagctccagtagctaaagtagcaccggtaagaactgtagcagcccctagagtggcaagtgttaaagtagtcactcctaaagtagaaactggtgcatcaccagagcatgtatcagctccagcagttcctgtgactacgacttcaacagctacagacagtaagttacaagcgactgaagttaagagcgttccggtagcacaaaaagctccaacagcaacaccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcaggtgatccaggtgatcatggtaaaggtttagcagtcgactttattgtaggtaaaaaccaagcacttggtaatgaagttgcacagtactctacacaaaatatggcagcaaataacatttcatatgttatctggcaacaaaagttttactcaaatacaaatagtatttatggacctgctaatacttggaatgcaatgccagatcgtggtggcgttactgccaaccattatgaccatgttcacgtatcatttaacaaataa

SEQ ID NO: 8 - protein, 409 aa, Streptococcus agalactiae
QETDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVSVADQKVSLNTISEGMTPEAATTIVSPMKTYSSAPALKSKEVLAQEQAVSQAAANEQVSTAPVKSITSEVPAAKEEVKPTQTSVSQSTTVSPASVAAETPAPVAKVAPVRTVAAPRVASVKVVTPKVETGASPEHVSAPAVPVTTTSTATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNK

SEQ ID NO: 9 - DNA, 1233 bp, Streptococcus agalactiae
caagaaacagatacgacgtggacagcacgtactgtttcagaggtaaaggctgatttggtaaagcaagacaataaatcatcatatactgtgaaatatggtgatacactaagcgttatttcagaagcaatgtcaattgatatgaatgtcttagcaaaaattaataacattgcagatatcaatcttatttatcctgagacaacactgacagtaacttacgatcagaagagtcatactgccacttcaatgaaaatagaaacaccagcaacaaatgctgctggtcaaacaacagctactgtggatttgaaaaccaatcaagtttctgttgcagaccaaaaagtttctctcaatacaatttcggaaggtatgacaccagaagcagcaacaacgattgtttcgccaatgaagacatattcttctgcgccagctttgaaatcaaaagaagtattagcacaagagcaagctgttagtcaagcagcagctaatgaacaggtatcaacagctcctgtgaagtcgattacttcagaagttccagcagctaaagaggaagttaaaccaactcagacgtcagtcagtcagtcaacaacagtatcaccagcttctgttgccgctgaaacaccagctccagtagctaaagtagcaccggtaagaactgtagcagcccctagagtggcaagtgttaaagtagtcactcctaaagtagaaactggtgcatcaccagagcatgtatcagctccagcagttcctgtgactacgacttcaacagctacagacagtaagttacaagcgactgaagttaagagcgttccggtagcacaaaaagctccaacagcaacaccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcaggtgatccaggtgatcatggtaaaggtttagcagtcgactttattgtaggtaaaaaccaagcacttggtaatgaagttgcacagtactctacacaaaatatggcagcaaataacatttcatatgttatctggcaacaaaagttttactcaaatacaaatagtatttatggacctgctaatacttggaatgcaatgccagatcgtggtggcgttactgccaaccattatgaccatgttcacgtatcatttaacaaatgttaa

SEQ ID NO: 10 - protein, 410 aa, Streptococcus agalactiae
QETDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVSVADQKVSLNTISEGMTPEAATTIVSPMKTYSSAPALKSKEVLAQEQAVSQAAANEQVSTAPVKSITSEVPAAKEEVKPTQTSVSQSTTVSPASVAAETPAPVAKVAPVRTVAAPRVASVKVVTPKVETGASPEHVSAPAVPVTTTSTATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNKC

SEQ ID NO: 11 - DNA, 1233 bp, Streptococcus agalactiae
caagaaacagatacgacgtggacagcacgtactgtttcagaggtaaaggctgatttggtaaagcaagacaataaatcatcatatactgtgaaatatggtgatacactaagcgttatttcagaagcaatgtcaattgatatgaatgtcttagcaaaaattaattgcattgcagatatcaatcttatttatcctgagacaacactgacagtaacttacgatcagaagagtcatactgccacttcaatgaaaatagaaacaccagcaacaaatgctgctggtcaaacaacagctactgtggatttgaaaaccaatcaagtttctgttgcagaccaaaaagtttctctcaatacaatttcggaaggtatgacaccagaagcagcaacaacgattgtttcgccaatgaagacatattcttctgcgccagctttgaaatcaaaagaagtattagcacaagagcaagctgttagtcaagcagcagctaatgaacaggtatcaacagctcctgtgaagtcgattacttcagaagttccagcagctaaagaggaagttaaaccaactcagacgtcagtcagtcagtcaacaacagtatcaccagcttctgttgccgctgaaacaccagctccagtagctaaagtagcaccggtaagaactgtagcagcccctagagtggcaagtgttaaagtagtcactcctaaagtagaaactggtgcatcaccagagcatgtatcagctccagcagttcctgtgactacgacttcaacagctacagacagtaagttacaagcgactgaagttaagagcgttccggtagcacaaaaagctccaacagcaacaccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcaggtgatccaggtgatcatggtaaaggtttagcagtcgactttattgtaggtaaaaaccaagcacttggtaatgaagttgcacagtactctacacaaaatatggcagcaaataacatttcatatgttatctggcaacaaaagttttactcaaatacaaatagtatttatggacctgctaatacttggaatgcaatgccagatcgtggtggcgttactgccaaccattatgaccatgttcacgtatcatttaacaaatgttaa

SEQ ID NO: 12 - protein, 410 aa, Streptococcus agalactiae
QETDTTWTARTVSEVKADLVKQDNKSSYTVKYGDTLSVISEAMSIDMNVLAKINCIADINLIYPETTLTVTYDQKSHTATSMKIETPATNAAGQTTATVDLKTNQVSVADQKVSLNTISEGMTPEAATTIVSPMKTYSSAPALKSKEVLAQEQAVSQAAANEQVSTAPVKSITSEVPAAKEEVKPTQTSVSQSTTVSPASVAAETPAPVAKVAPVRTVAAPRVASVKVVTPKVETGASPEHVSAPAVPVTTTSTATDSKLQATEVKSVPVAQKAPTATPVAQPASTTNAVAAHPENAGLQPHVAAYKEKVASTYGVNEFSTYRAGDPGDHGKGLAVDFIVGKNQALGNEVAQYSTQNMAANNISYVIWQQKFYSNTNSIYGPANTWNAMPDRGGVTANHYDHVHVSFNKC

SEQ ID NO: 13 - DNA, 38 bp, Artificial Sequence
  Description of Artificial Sequence Synthetic primer
ccggcagatctatgaaaatgaataaaaaggtactattg

SEQ ID NO: 14 - DNA, 36 bp, Artificial Sequence
  Description of Artificial Sequence Synthetic primer
ggccgtctagattatttgttaaatgatacgtgaaca

SEQ ID NO: 15 - DNA, 45 bp, Artificial Sequence
  Description of Artificial Sequence Synthetic primer
cacgtatcatttaacaaatgttaatctagagcttgaggatccggc

SEQ ID NO: 16 - DNA, 45 bp, Artificial Sequence
  Description of Artificial Sequence Synthetic primer
gccggatcctcaagctctagattaacatttgttaaatgatacgtg

SEQ ID NO: 17 - DNA, 41 bp, Artificial Sequence
  Description of Artificial Sequence Synthetic primer
gaatgtcttagcaaaaattaattgcattgcagatatcaatc

SEQ ID NO: 18 - DNA, 41 bp, Artificial Sequence
  Description of Artificial Sequence Synthetic primer
gattgatatctgcaatgcaattaatttttgctaagacattc

SEQ ID NO: 19 - DNA, 45 bp, Artificial Sequence; identical to SEQ ID NO: 15
  Description of Artificial Sequence Synthetic primer

SEQ ID NO: 20 - DNA, 45 bp, Artificial Sequence; identical to SEQ ID NO: 16
  Description of Artificial Sequence Synthetic primer